(19) United States
(12) Patent Application Publication
(10) Pub. No.: US 2014/0243359 A1
(43) Pub. Date: Aug. 28, 2014

(54) ADENINE DERIVATIVES HAVING IMMUNOMODULATING ANTI-INFLAMMATORY AND ANALGESIC ACTIVITY

(71) Applicants: Giulia Federica Merizzi, Torino (IT); Fabio Grassi, Bellinzona (CH); Antonio Soleti, Nichelino (IT)

(72) Inventors: Giulia Federica Merizzi, Torino (IT); Fabio Grassi, Bellinzona (CH); Antonio Soleti, Nichelino (IT)

(73) Assignee: MEDESTEA RESEARCH & PRODUCTION S.P.A., Colleretto Giacosa, Torino (IT)

(21) Appl. No.: 14/233,344

(22) PCT Filed: Jul. 20, 2012
(86) PCT No.: PCT/IB2012/053706

§ 371 Date: Jan. 16, 2014

(30) Foreign Application Priority Data
Jul. 20, 2011 (IT) TO2011A000652

(51) Int. Cl. A61K 31/52; A61K 31/337; A61K 31/485; C07D 473/34

(52) U.S. Cl. CPC A61K 31/52; C07D 473/34; A61K 31/337; A61K 31/485
USPC 514/263.23; 544/277; 514/263.4

(57) ABSTRACT

The compound 2-[1-(6-aminopurin-9-yl)-2-oxoethoxy]prop-2-enal of formula (I), its monohydrate of formula (II), or its corresponding cyclic monohydrate of formula (III), in which the stereochemical chiral center indicated with an asterisk can be R (Rectus), S (Sinister) or racemic, including the tautomers of the adenine ring and the pharmaceutically acceptable salts, are used in the treatment of inflammatory disorders or of pain disorders.

The present invention relates to novel therapeutic uses of the compound 2-[1-(6-aminopurin-9-yl)-2-oxoethoxy]prop-2-enal having the formula:

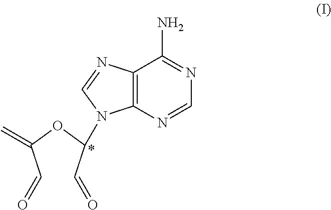 [SMILES: C=C(C=O)OC(C=O)n1cnc2c(N)ncnc21]

of its corresponding monohydrate of formula (II)

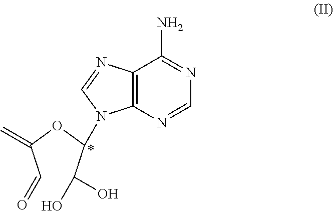 [SMILES: C=C(C=O)OC(C(O)O)n1cnc2c(N)ncnc21]

and of its cyclic monohydrate of formula (III)

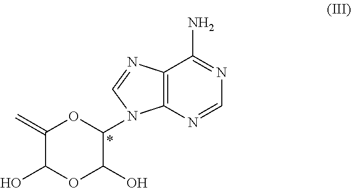 [SMILES: C=C1OC(n2cnc3c(N)ncnc32)C(O)OC1O]

The scope of the invention includes the use of the aforementioned compounds, both in racemic form, and in the form of their R and S optical isomers, with respect to the chiral centre identified with an asterisk in the formulae given above; the use of the tautomers of the adenine ring and of the pharmaceutically acceptable salts is also included.

The compound of formula (I) is known from the literature in its racemate form (CAS 110326-59-5) and as the R enantiomer (CAS 70512-83-3) and S enantiomer (CAS 73566-63-9). Compound (I) can be prepared by the method described by Grant et al. in Journal of Medicinal Chemistry (1980), 23(7)795-8, by oxidation of the nucleoside of adenine with periodate, to obtain the dialdehyde of the nucleoside and then application of heat to the aqueous solution of the nucleoside dialdehyde.

Alternatively, the compound can be prepared by oxidation of adenosine monophosphate (AMP) with periodate as described in the preparative example given below.

For this compound, cytotoxic activity on the murine neuroblastoma in tissue cultures has already been reported in the literature (B. L. Mirkin et al. in Cancer Research 47, 3650-3655, Jul. 15, 1987).

The corresponding monohydrate (II) is also known in the form of racemate (CAS 82422-43-3) and of R enantiomer (CAS 149585-92-2).

The cyclic monohydrate (III) is not described in the literature; however, its presence can be presumed in view of the tautomeric equilibrium with the monohydrate of formula (II).

The various stereoisomers of 1,4-dioxane-3,5-diols of the cyclic monohydrate (III), including in particular the 3,5-diaxial stereoisomer, are also covered by the invention.

The object of the invention is defined by the claims given hereunder, which form part of the present description.

Tests of biological activity, carried out in the context of the research that led to the invention, demonstrated that the aforementioned compounds display high anti-inflammatory, immunosuppressive, analgesic and angiogenesis-inhibiting activity.

In view of the analgesic and anti-inflammatory activity, the compounds can be used for treating pain pathology of various origins, such as nociceptive pain (somatic, osteoarticular and visceral), neuropathic pain and cancer pain; in particular, the compounds have demonstrated high activity in the treatment of pain following the administration of chemotherapeutic agents, in particular paclitaxel, so that the compounds can be used in combination therapy for treating pain caused by chemotherapeutic compounds. The compounds have also demonstrated a synergistic effect in potentiation of the analgesic effect of opioids, such as morphine and the like.

Owing to the anti-inflammatory activity, the compounds may prove useful in disorders of varying aetiology, but where the inflammatory component plays a significant role in the symptomatology, such as muscular dystrophy, uveitis, asthma or Alzheimer's disease.

Owing to the anti-inflammatory and angiogenesis-inhibiting activity, the compounds can be used in the treatment of eye disorders that include intensive vascularization, such as diabetic retinopathy, macular degeneration, proliferative vitreoretinopathy, glaucoma, as well as in the treatment of arteriosclerotic processes.

Experimental tests, in vitro and in vivo, further demonstrate that these compounds, in addition to their potent anti-inflammatory and analgesic action, also possess immunosuppressive and immunomodulatory activity that promotes the generation and proliferation of a lymphocyte population of the T-regulatory (T_{reg}) type, lymphocytes that have been designated to contain the body's immune response. The compounds also have inhibitory action on the maturation of dendritic cells. The compounds can therefore be used in the treatment of inflammatory disorders that are dependent on imbalance of the immune response (including autoimmune diseases), for example COPD, cystic fibrosis, rheumatoid arthritis, type I and II diabetes, systemic lupus erythematosus (SLE), multiple sclerosis, amyotrophic lateral sclerosis, scleroderma, dermatomyositis, Sjögren polymyositis syndrome, Erdheim-Chester syndrome, psoriasis, inflammatory bowel disease (IBD), Crohn's disease, Takayasu disease, in transplant rejection, graft-versus-host disease.

For therapeutic uses, the compounds can be formulated with pharmaceutically acceptable excipients and carriers and administered by the oral, topical, parenteral, inhalational and rectal route.

The pharmaceutical forms, usable by different routes of administration, comprise tablets, pills, capsules (including soft-gel), granules, powders, syrups, suspensions, creams, suppositories, gels, pastes, ointments, lotions, emulsions, sprays, aerosols, puffs and every other form usable in administration of the drug.

The pharmaceutical composition can be prepared as described in Remington's Pharmaceutical Sciences Handbook, Mack Pub. Co., NY, USA, XVII Ed.

The amount of active substance per daily dose is in the range from 0.001 to 100 mg per kg of body weight, preferably from 0.1 to 20 mg, depending on the type and severity of the disease. In general, the unit dose for administration could vary between 3 and 500 mg and is preferably between 5 and 300 mg.

The present invention further relates to combined pharmaceutical preparations of the aforementioned compounds and other biologically active substances in the treatment of transplant rejection, in autoimmune disorders, and in pain and inflammatory syndromes.

In one embodiment of the invention, the compounds can be used in combination with antitumour substances such as alkaloids, antibiotics, cytotoxic and cytostatic compounds, antimetabolites, antihormonal agents, alkylating agents, peptides, modulators of the biological response, cytokines.

The compounds can be used in combination with immunosuppressive drugs (ciclosporin, sirolimus, tacrolimus, everolimus, methotrexate, as a non-exhaustive list) or monoclonal antibodies for the treatment of autoimmune disorders or of transplant rejection, or with other drugs used for treating inflammation, such as NSAIDs, corticosteroids or antioxidants, as a non-exhaustive list.

The various active substances can be administered both simultaneously and separately.

The choice of the specific combination of pharmacologically active substances, their dosage and the route of administration depends on the type of pathology and its resistance to drug treatment and can be modified case by case on the basis of the patient's tolerance and other variables.

In the present description, the compound of formula (I) is identified with the term IMMUFLAM.

### PREPARATIVE EXAMPLE

### Compound of Formula (I)

The following synthesis protocol is followed: dissolve 1 equivalent of AMP in water (to a concentration of about 0.3M), adjust the solution pH to 8.4 with NaOH (2M). Add 1 equivalent of NaIO₄ to the mixture. Maintain the reaction at room temperature for 5 minutes and then at 45° C. for three hours. Then carry out silica chromatography, after removing the water from the initial solution by lyophilization. Dissolve the crude lyophilized product in chloroform and methanol, mix with silica, and dry the mixture and load it in the top of the silica column. Elute the column with 95/5 chloroform/methanol solution, recovering the desired product at high purity.

### 1. Anti-Inflammatory Effect: Demonstration In Vitro

It was demonstrated in vitro that IMMUFLAM reduces the secretion of pro-inflammatory cytokines, after stimulation with LPS (pro-inflammatory stimulus) in U937 cells, an immortalized monocytic cell line from histiocytic lymphoma.

The production in vitro of MCP-1 (monocyte chemoattractant protein-1), TNF-α (tumour necrosis factor alpha), IL-6 (interleukin 6) was analysed. These cytokines induce the production of other cytokines that attract leukocytes to the site of inflammation, potentiating the immune response. This amplifies leukocyte “recruitment” at the site of inflammation, promoting “extravasation” of immune cells from the peripheral circulation.

The U937 cells were plated at a density of 250 000 cells/ml and left in quiescence for 24 hours.

The drug was added in fresh medium, simultaneously or after removal of LPS from the culture. The cells were kept in an incubator at 37° C. with supply of CO₂ at 5%.

Analysis of the production of growth factors was performed on the cellular supernatants collected, by ELISA assay using commercial kits (Human TNF-α Quantikine ELISA Kit code DTA00C, R&D Systems assay. Human IL-6 Quantikine ELISA Kit code D6050, R&D Systems assay. Human CCL2/MCP-1 Quantikine ELISA Kit code DCP00, R&D Systems assay).

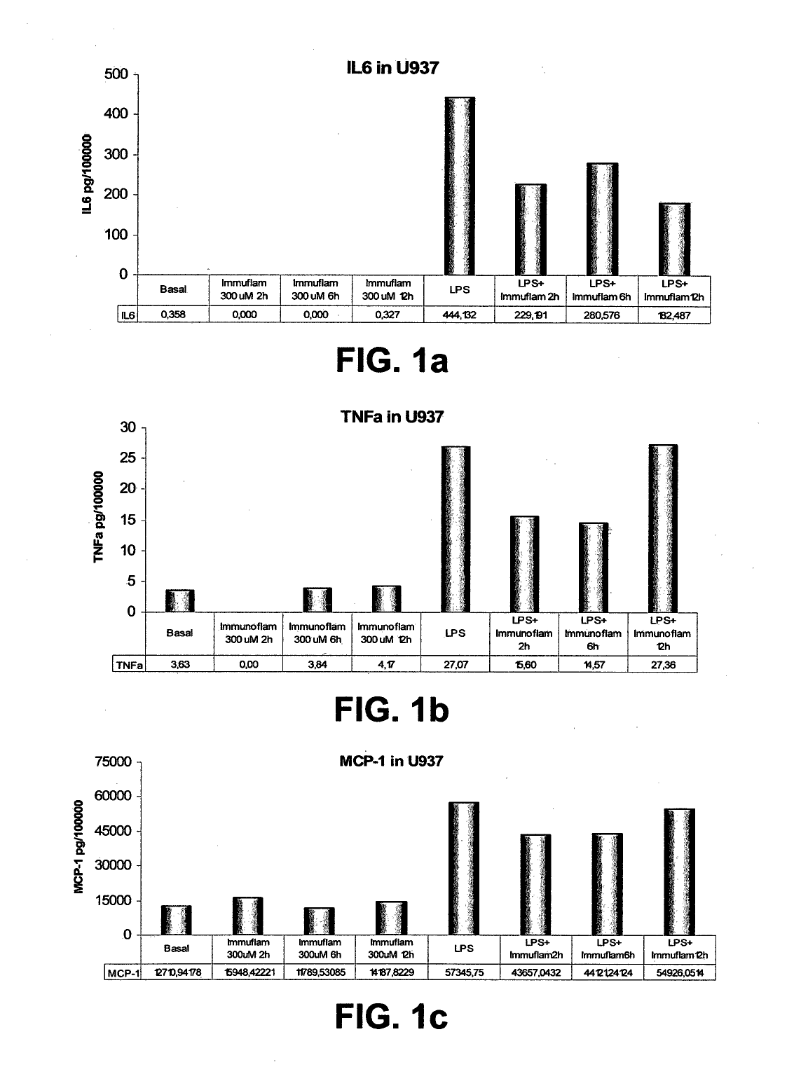

The results presented in the bar charts in FIG. 1a-c clearly show that IMMUFLAM reduces the cellular secretion of the cytokines analysed both in the presence of the inflammatory stimulus LPS (12 hrs) and when it is added after the inflammatory stimulus for 2 or 6 hours.

### 2. Antiangiogenic Effect: Inhibition of Secretion of VEGF

The inhibitory action of IMMUFLAM on the production of VEGF in the supernatant of U937 in culture was also evaluated.

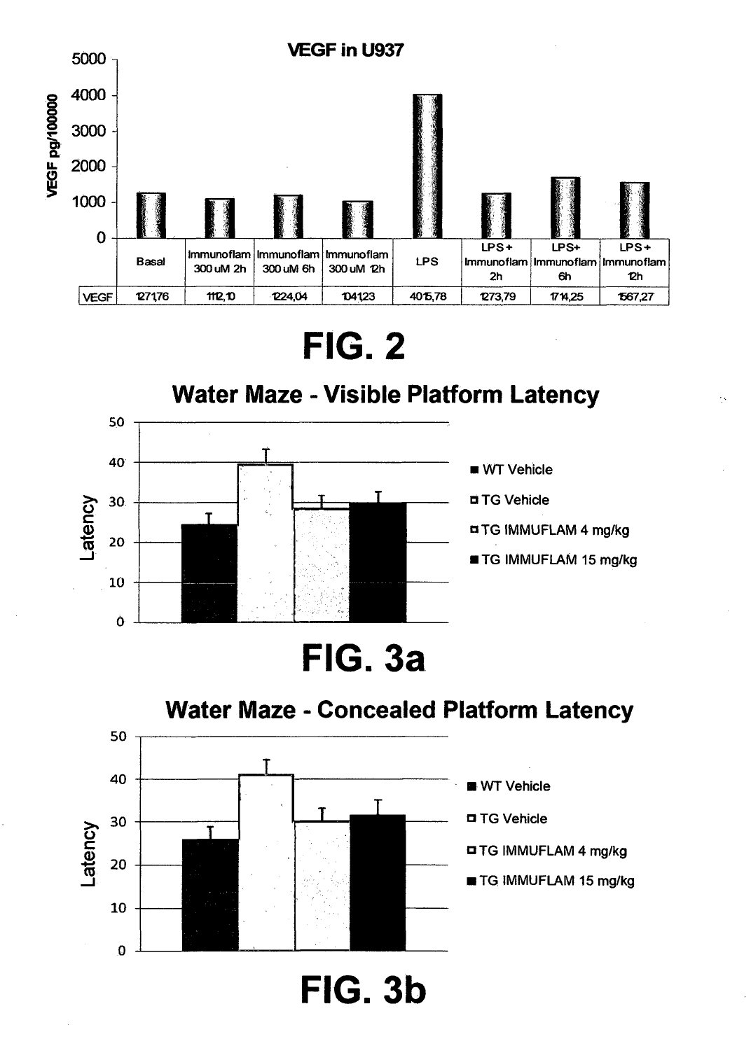

As shown by the bar chart in FIG. 2, IMMUFLAM reduced the cellular production of VEGF significantly.

### 3. Alzheimer's

Alzheimer's is a progressive neurodegenerative disorder characterized by loss of memory and of major cognitive functions. The distinctive neuropathologic features are the accumulation, up to formation of plaques, of β-amyloid peptides, activation of the glial cells close to the plaques, a consequent increase in inflammatory cytokines and loss of neurons in some specific zones of the brain.

In the transgenic murine model Tg2576 there is no loss of neurons, but there is neuronal deficit and accumulation of β-amyloid plaques. The production of peptides begins to increase rapidly starting from the 6th month of age, accompanied by a progressive cognitive decline. Around the 7th to 8th month, the first plaques develop, which are then diffuse at the 12th month.

The mice were treated with IMMUFLAM at 4 and at 15 mg/kg i.p. daily starting from the 6th month of age up to the 13th. Between the 9th and 10th month and between the 12th and 13th month the mice were submitted to behavioural tests, at the 13th month they were sacrificed and immunohistochemical analyses were performed on brain samples to verify modulation of the β-amyloid plaques.

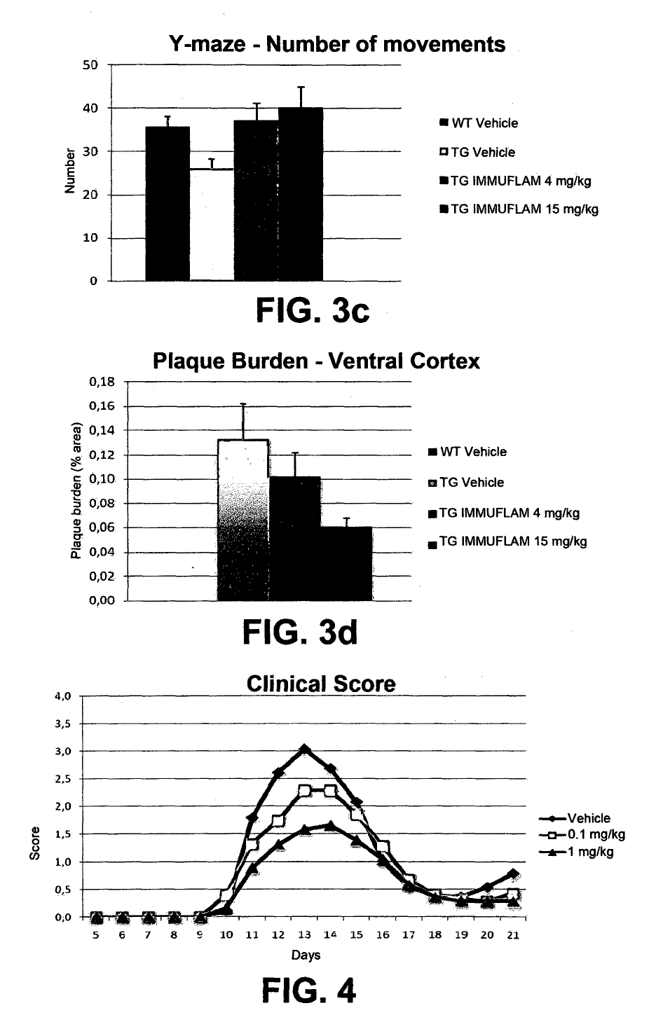

The behavioural tests revealed potent action of IMMUFLAM even at the dosage of 4 mg/kg. The effect is found to be independent of dose and the difference from the control is statistically significant with both dosages. The mice succeeded in finding and climbing onto the platform, hidden or visible, significantly faster than the control treated with the vehicle. At the same time the score obtained in moving to Y was very encouraging. IMMUFLAM significantly reduced soluble plaques compared with the control that only received the vehicle. In this case a dose-dependent effect was observed. The results of the tests are presented in FIGS. 3a-d.

4. Improvement of the Symptoms of Acute Experimental Autoimmune encephalomyelitis (EAE)

We investigated whether treatment with IMMUFLAM, at the onset of the acute form of experimental autoimmune encephalitis (EAE), the murine model of human multiple sclerosis, could induce a functional recovery.

The motor and sensory deficits were classified according to previously assigned “neuronal” clinical scores.

Adult female Lewis rats (200-250 g) were immunized with an intradermal injection, in the hind limbs, of 100 mg of myelin basic protein (MBP) dissolved in 50 microlitres of emulsified saline solution in 50 microlitres of complete Freund adjuvant (CFA).

Starting from the fifth day post-inoculation, the rats were treated daily with IMMUFLAM (0.1 or 1 mg/kg per os) or with the placebo (vehicle).

The animals were monitored daily until day 21 for clinical symptoms; the range of neuroclinical scores extended from 0 (no neurologic symptom) up to a maximum of 5 (complete paralysis of all legs or death).

Inoculation with the MBP/CFA emulsion led to a very evident loss of body weight; this was not changed by the treatment with Immuflam. The animals were divided into three groups of 15: group 1 Immuflam 0.1 mg/kg, group 2 Immuflam 1 mg/kg, group 3 vehicle. They were all treated daily per os from day 5 until sacrifice.

### Results

Starting from days 10-11, the onset of acute EAE caused a significant deterioration of the clinical scores, with a peak on day 13. In the intervening period between day 12 and day 15, analysis of the clinical “scores” revealed significant differences between the groups treated with the vehicle and the groups treated with IMMUFLAM (FIG. 4).

The peak of neurologic damage for group 3 was found to be equal to 3 (day 13), in group 1, the peak decreases to 2.3 while in group 2 to 1.5.

The results show that IMMUFLAM, administered daily, has a beneficial effect on functional recovery in the subjects with acute EAE compared to placebo, and this effect is dose-dependent.

### 5. Improvement of the Symptoms of Chronic Experimental Autoimmune Encephalomyelitis (EAE)

We investigated whether treatment with IMMUFLAM, at the onset of EAE induced by immunization with MOG (myelin-oligodendrocyte-glycoprotein), stimulates histologic protection and functional recovery.

The motor and sensory deficits were classified according to the previously assigned “neuronal” clinical scores.

Chronic EAE was induced in 60 female adult Dark Agouti (DA) rats of 120-170 g, immunizing the animals with an intradermal injection, at the base of the tail, of 50 mg of recombinant protein MOG1-125 in emulsified PBS in complete Freund adjuvant (CFA) containing hot-inactivated M. tuberculosis.

The rats were divided into 3 groups of 20 animals each and were treated with daily administrations of vehicle or IMMUFLAM 0.1 or 1 mg/kg per os, starting from the day of inoculation.

The neurological scores were measured and evaluated daily for the first thirty days, and thereafter only on weekdays (Monday-Friday) until the end of the study (day 50).

The range of neuroclinical scores begins at 0 (absence of neurologic symptoms) and ends with a score of 5 (complete paralysis of all legs or animal moribund).

### Results

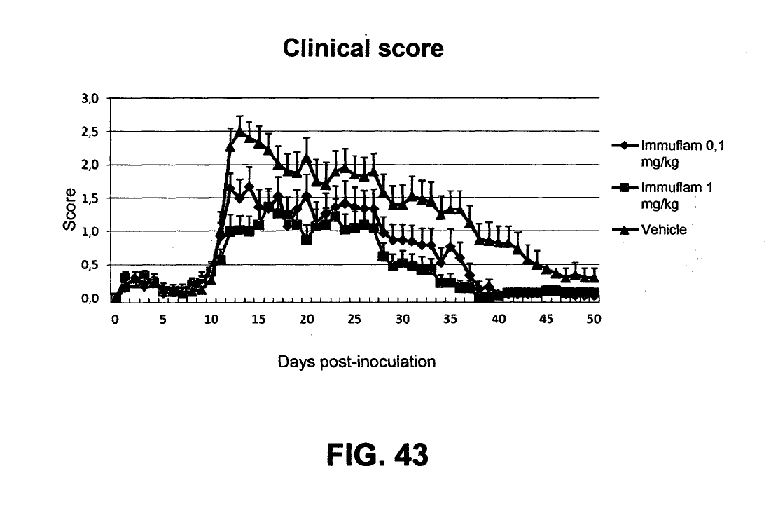

Inoculation of the MBP/CFA emulsion led to a drop in weight starting from the tenth day after injection, which was then recovered around day 17. Counting of the neurological scores identified a clear worsening of the groups on day 10, which reached the peak on day 13 in the vehicle group, whereas for the group treated with 1 mg/kg of Immuflam, the scores remained lower throughout the period of observation, as shown in FIG. 43. The effect of Immuflam is found to be dose-dependent, with the group that received the highest dose (1 mg/kg) which has a very low score from the start. The results suggest that Immuflam can significantly improve the neurological scores of the chronic murine model of multiple sclerosis.

### 6. Ischaemia/Reperfusion Injury

Ischaemia is an acute pathologic event in which there is reduction in blood supply with resultant injury or dysfunction of the affected tissue. Currently, the obstructed blood vessel is cleared by direct intervention. The resumption of normal circulation after a period of hypoxia causes quite considerable tissue injury, however, with fibrotic results that undermine the functionality of the affected organ. This has been demonstrated at the cardiac, cerebral and renal level. We wanted to check whether IMMUFLAM could reduce the injury due to reperfusion and to do so we used a model of renal ischaemia. The model envisages creation of an ischaemic event and then verification of the injury following treatment with IMMUFLAM or with the vehicle.

The experiment was conducted on Sprague-Dawley (SD) rats of 250-300 g divided into three groups. The first group, the positive control, only underwent an incision, which was kept open for 30 minutes, without ischaemic injury. The other two were pretreated, 30 minutes before undergoing the intervention, with intraperitoneal administration of vehicle (group 2) or of IMMUFLAM (group 3)10 mg/kg. The rats were anaesthetized and, after making an abdominal incision, the arteries and veins were occluded bilaterally with a microvascular clamp. After occlusion for 30 minutes, the clamps were removed and the incision was closed. The rats were left at rest for 24 hours and then were sacrificed, collecting a blood sample for determination of creatinine by the Jaffe reaction, as evaluation of renal injury. Both kidneys were stored for histologic examination.

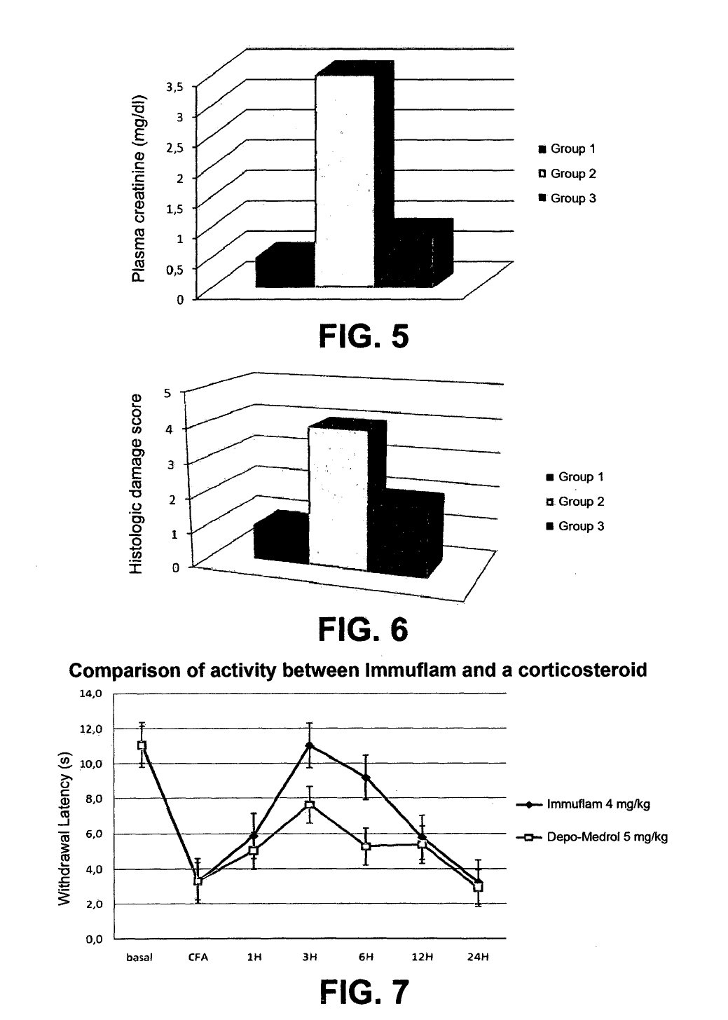

As can be seen from the bar chart in FIG. 5, group 1 has normal plasma creatinine levels, group 2 has a dramatic increase in these levels, while group 3 shows values much closer to those of group 1.

The kidneys were sectioned for histologic analysis in double-blind conditions. The percentages of the tissue changes observed were counted using a 5-point semiquantitative scale, defined for assessing necrosis and cellular loss: 0=normal kidney; 0.5=<10%; 1=10-25%; 2=25-50%; 3=50-75%; 4=75-100%.

It was found that IMMUFLAM largely prevents renal injury due to ischaemia and reperfusion, both on assessing the renal tissue and on assessing organ functionality (FIG. 6).

### 7. Anti-Inflammatory and Analgesic Effect In Vivo: Model of Arthritis

IMMUFLAM was tested for possible antinociceptive and anti-inflammatory activity in a murine model of arthritis.

Unilateral inflammation of the posterior plantar pad of the rat (male Fischer inbred rats) was induced with a local injection of complete Freund adjuvant. Administration of the adjuvant led to a significant increase in measured nociception and a decrease in withdrawal time relative to baseline measurements taken in accordance with Hargreaves' method (plantar test).

IMMUFLAM, 4 mg/kg, was administered intraperitoneally.

The rats were put in a transparent plastic chamber and were left to acclimatize for 5 minutes before the test.

Thermal stimulus, applied to the surface of the plantar pads of the rats, was produced by a halogen lamp (64607 OSRAM) of 8 V-50 W, through the plastic box; the diameter of the radiant beam measured about 12 mm. The time for withdrawal of the animal's right plantar pad was measured before and after injection of IMMUFLAM.

A comparison was conducted on the anti-hyperalgesic and anti-inflammatory effect of a cortisone derivative, methylprednisolone acetate, administered intraperitoneally (5 mg/kg). The comparative results are presented in FIG. 7.

In conclusion, IMMUFLAM significantly increases the time for withdrawal of the plantar pads, bringing it to levels comparable to the baseline values, showing a more effective action relative to one of the drugs most used for pain and inflammation.

The data indicate that, in the course of peripheral inflammation, IMMUFLAM reduces nociceptive perception.

### 8. Anti-Inflammatory and Analgesic Effect: Acute Model

The role of IMMUFLAM was investigated by inducing inflammation of the plantar pads of rats with carrageenan, a model that mimics the state of acute inflammation in humans.

The results obtained showed that local and intravenous administration of a single dose of IMMUFLAM (6 mg/kg) significantly reduces thermal hyperalgesia in the plantar pads of rats for 24 hours and this effect was found to be greater than the effect produced by diclofenac or indometacin.

Inflammation was induced in male Wistar rats by means of injection of 0.1 ml of carrageenan at 1% in sterile saline solution in the rats' right plantar pad. Peak inflammation occurs 3 hours after injection of carrageenan; at this point (t=0), the rats are treated with IMMUFLAM or diclofenac or indometacin (100 μM in 0.15 ml); the antinociceptive effect of the drugs was measured at 1, 3, 6, 12 and 24 hours after their administration.

The threshold value of nociception on thermal stimulus was measured with Hargreaves' method and biopsies from the plantar pads were submitted to immunohistochemical investigations, which showed that treatment with IMMUFLAM significantly reduces the presence of macrophages infiltrating the plantar pad.

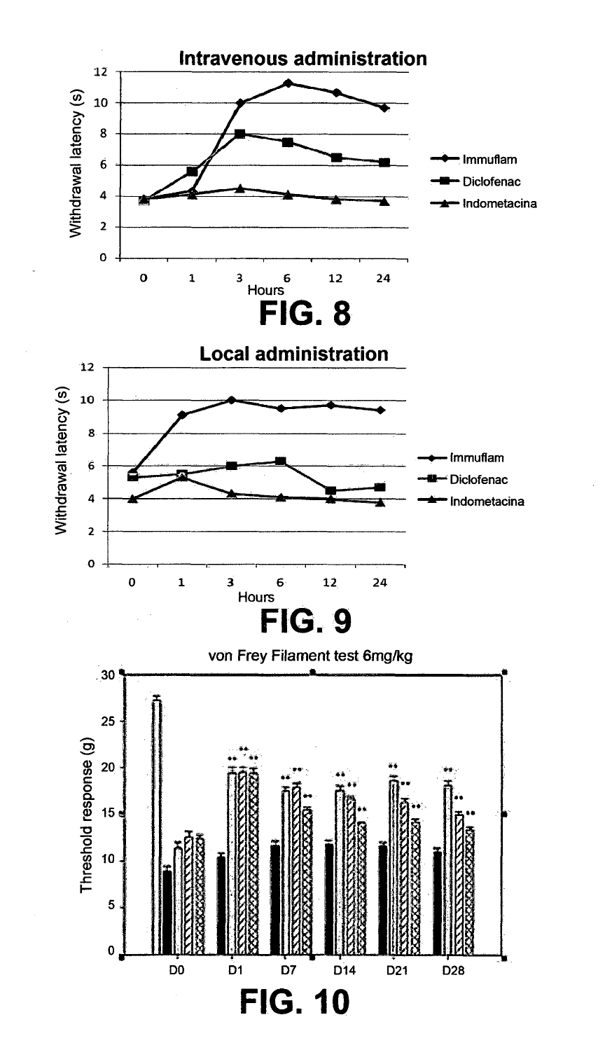

In conclusion, all the results obtained (FIG. 8 and FIG. 9) clearly show that IMMUFLAM reduces carrageenan-induced inflammation in rats.

### Example 12

### Visceral

### 9. Neuropathic Pain: SNI Model

We evaluated the effect of IMMUFLAM in a model of neuropathic pain: the model of lesion of the sciatic nerve. The characteristic feature of neuropathic pain is that both macroglia and microglia (the cells that support the neurons metabolically) can alter their phenotype (reactive gliosis) as a result of some traumatic-inflammatory peripheral events, duplicated in this model. These phenotypic and functional changes can become permanent and the perception of pain can continue even after its primary cause has disappeared. The cells react to the positive lesion by increasing the glial fibrillary acidic protein (GFAP) and the ionized calcium-binding adapter molecule (IBA1). Male Sprague-Dawley rats (250-300 g; Harlan, Italy) were divided into 5 groups.

Group I was treated with IMMUFLAM daily from day 1 to day 21; group II was treated daily with the drug from day 1 to day 7 and then again with a single dose on day 14; group III was treated with IMMUFLAM on day 1, day 7 and day 14; group IV was treated with only the vehicle and group V was submitted to a sham operation (positive control).

After isolation and exposure of the sciatic nerve, the three terminal branches were separated: the tibial nerve and the common peroneal nerve were ligated individually and cut in a position distal to the ligature. The sural nerve was left intact.

IMMUFLAM was administered intraperitoneally at 2 doses: 6 mg/kg and 30 mg/kg in DMSO.

Behavioural tests were performed daily from day 0 to day 7 and then on alternate days until day 28. Mechanical allodynia was induced by von Frey filament stimulation and the onset of thermal hyperalgesia was measured using the Ugo Basile apparatus according to Hargreaves' method, 1988.

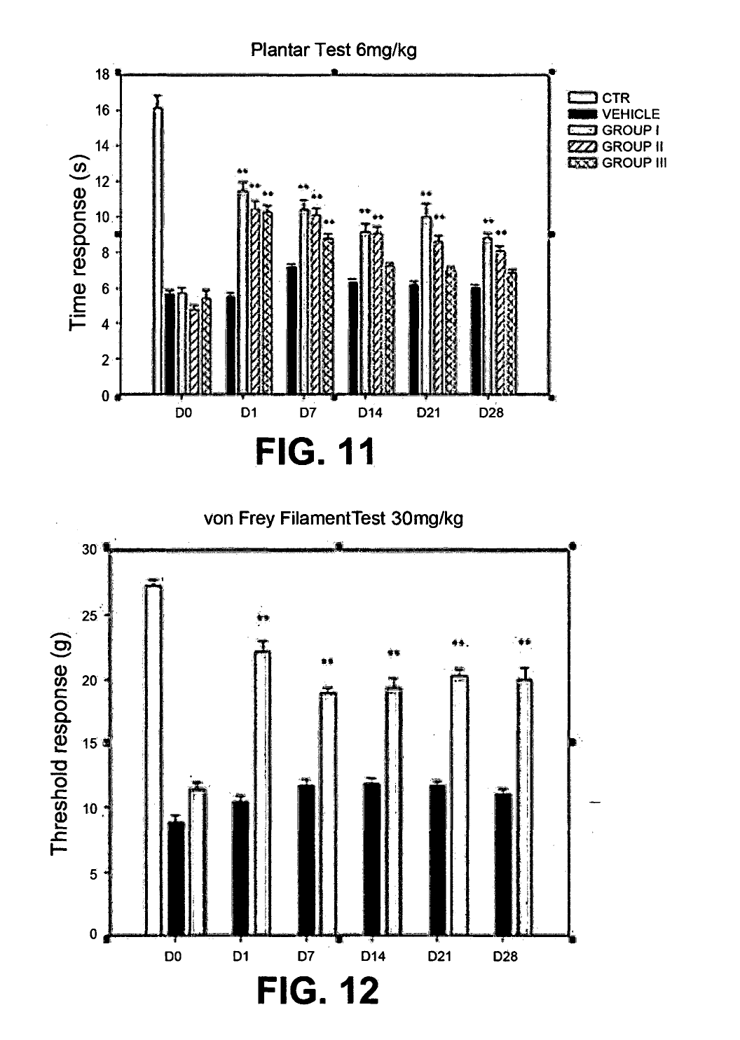

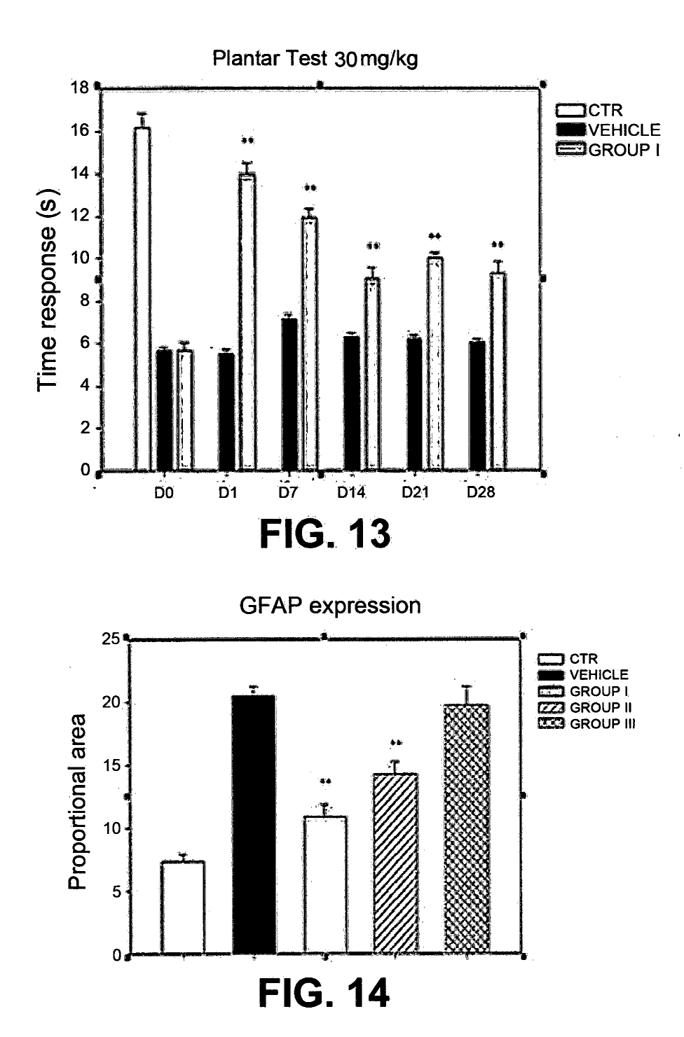

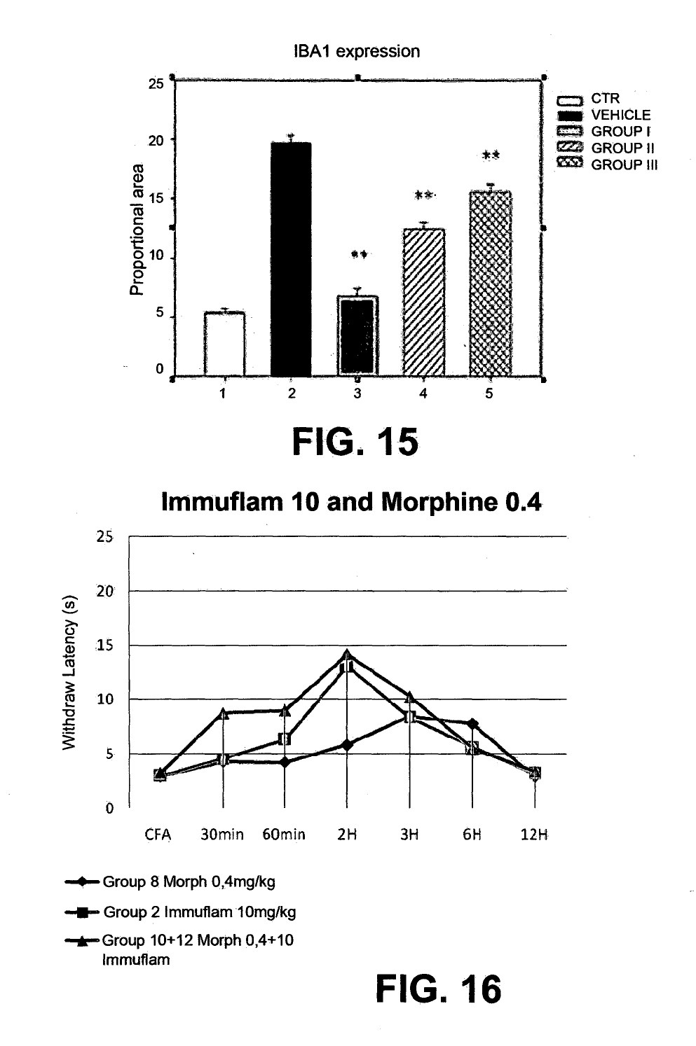

The results obtained are shown in the bar charts in FIGS. 10 to 15.

IMMUFLAM reduces pain significantly starting from the first day of administration at both doses and maintains this condition until 7 days after the end of the treatment, significantly even at the lower dose.

Surprisingly, in the animals treated daily with IMMUFLAM, expression of the proteins of reactive gliosis Iba1 and GFAP returns almost to the normal levels (immunohistochemical analysis). The compound indeed proved to be effective in the treatment of neuropathic pain both in the inflammatory component and in the hyperalgesic component.

10. Pain: Synergy with Opioids

We compared the activity of IMMUFLAM with that of the opioids to study its analgesic effect.

Mice of the Swiss CD1 type were given a single intraplantar injection of 30 microlitres of complete Freund adjuvant (CFA) in the right plantar pad. Intraperitoneal injection of IMMUFLAM and/or of morphine (morphine chlorhydrate, Molteni) was administered 24 hours after injection of CFA according to the following scheme:

### Groups:

1. IMMUFLAM 7.5 mg/kg; 2. IMMUFLAM 10 mg/kg; 3. IMMUFLAM 200 mg/kg; 4. IMMUFLAM 50 mg/kg; 5. Morphine 0.4 mg/kg; 6. Morphine 2.5 mg/kg; 7. Morphine 1 mg/kg; 8. Morphine 0.4 mg/kg; 9. Morphine 1 mg/kg+IMMUFLAM 10 mg/kg; 10. Morphine 0.4 mg/kg+IMMUFLAM 10 mg/kg; 11. Morphine 1 mg/kg+IMMUFLAM 10 mg/kg; 12. Morphine 0.4 mg/kg+IMMUFLAM 10 mg/kg; 13. Morphine 4 mg/kg.

Groups 9-11 and 10-12 have the same doses respectively; they were repeated to confirm the results.

Thermal hyperalgesia was assessed using the Ugo Basile apparatus (Comerio, Italy).

The test was carried out before (baseline values) injection of CFA, after injection of CFA and 0.5, 1, 2, 3, 6 and 12 hours after injecting the drug in the inflamed pad.

The mice treated with 2.5 mg/kg of morphine (or with higher doses) displayed obsessive-compulsive disorders. In contrast, the mice treated with IMMUFLAM did not display any behaviour of this kind.

### Results

The results show that, at all the doses tested, IMMUFLAM brings back the values of nociception to the baseline values. The activity of morphine at the highest dose is anaesthetic-like.

The combination of IMMUFLAM and low doses of morphine shows a significant improvement of PWL especially at a dose of 10 mg/kg of IMMUFLAM+1 mg/kg of morphine without obsessive-compulsive effects.

The combination of IMMUFLAM with morphine shows synergistic activity in pain reduction. These results justify the possibility of co-administration of the two drugs at low dose for reducing pain while avoiding the multiple side-effects typical of the opiates.

11. Pain from Administration of Chemotherapeutic Drugs: Model with Paclitaxel

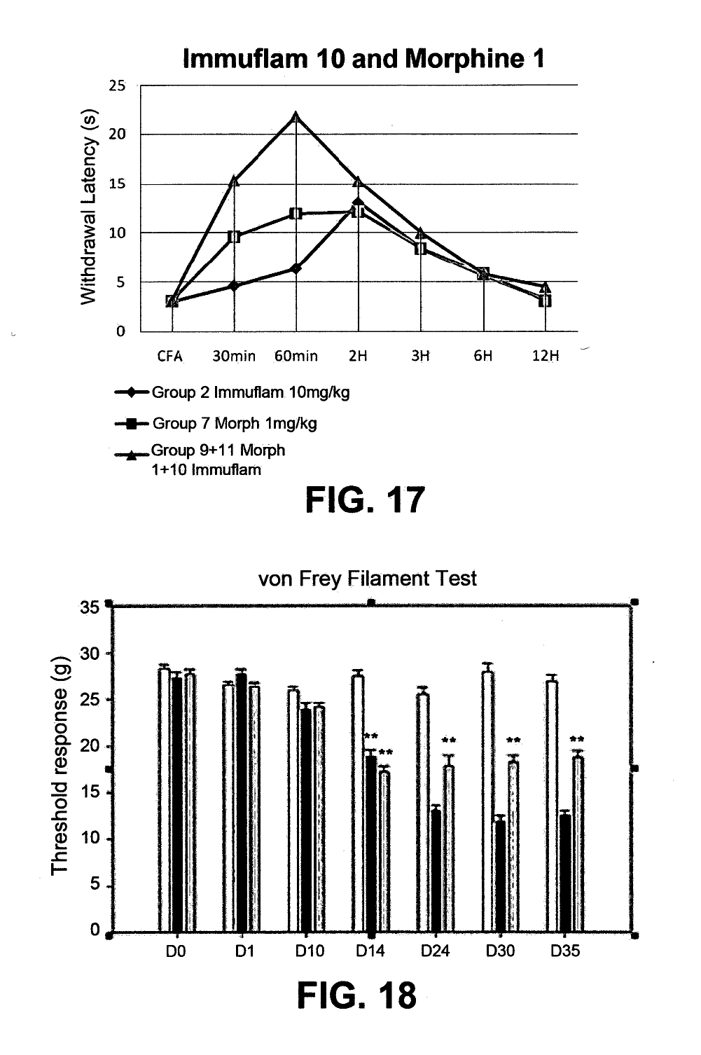

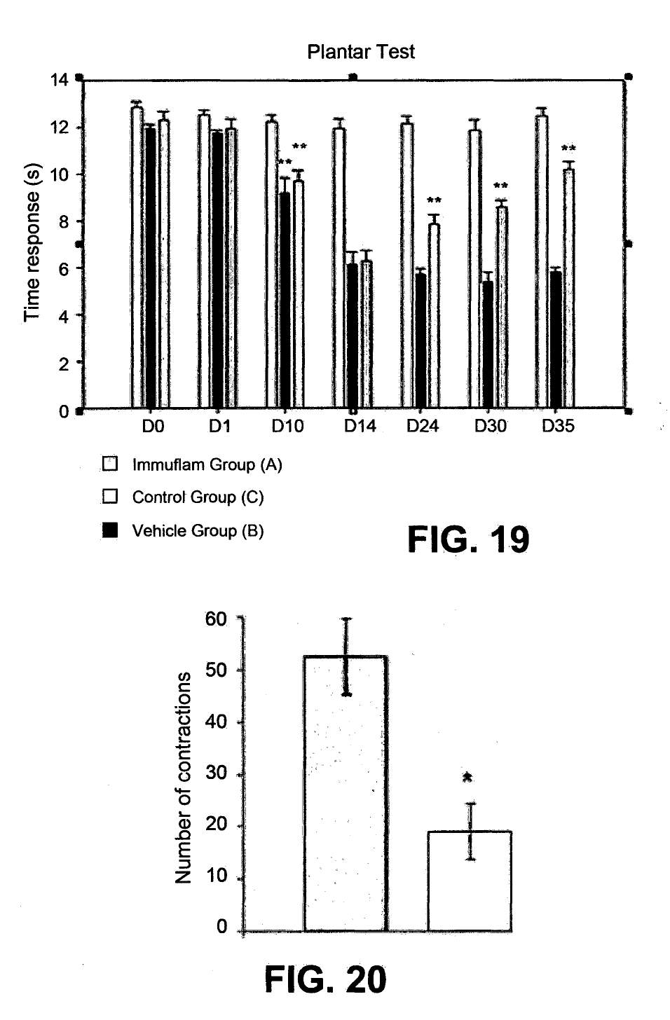

It has been demonstrated that repeated administration of chemotherapeutic agents for treatment of neoplasms increases the patient's pain state. One of the best validated models for testing this effect is that described hereunder; repeated administration of paclitaxel to rats. The animals (male Sprague Dawley rats weighing 250-300 g; Harlan, Italy) were treated with paclitaxel (2 mg/kg) administered intraperitoneally on alternate days (day 0, 2, 4, 6). The behavioural tests for thermal and mechanical sensitivity were carried out on day 1 (the day following the first injection of paclitaxel), day 10, day 14 (7 days from the last injection), day 24 (peak severity of hyperalgesia and allodynia), day 30 and day 35 (day on which the animals were sacrificed). The animals (n=15) were divided into three experimental groups. The first group (A) received the treatment with paclitaxel, and was treated daily with IMMUFLAM, 6 mg/kg by intraperitoneal injection from day 14 to day 35; the second group (B) treated with paclitaxel, was treated from day 14 to 35 with only the vehicle, and finally the third group consisted of naive animals (C). The results obtained are shown in the bar charts in FIGS. 18 and 19.

Pain associated with administration of paclitaxel appears starting from day 14 (one week after the last injection) and reaches peak severity on days 20-25. On day 14, the animals were divided into groups based on the degree of mechanical hypersensitivity and assigned randomly to the IMMUFLAM groups or the vehicle group. The rats treated with paclitaxel, and vehicle, display a significant state of allodynia and hyperalgesia throughout the study (days 35). The mean value of the baseline test of normal mechanical resistance, recorded before administration of paclitaxel (day 0), was equal to 28.35±0.43 s in the naive animals (control); this value remained almost constant. On day 10 the rats treated with paclitaxel show a lower threshold relative to the controls. The difference becomes significant (P≦0.001) starting from day 14 (27.52±0.58 s in group C, 18.87±0.73 s in group B and 17.22±0.62 s in group A). A significant reduction of the nociceptive threshold to the von Frey filament test appears starting from day 24, until the end of the experiment. The intraperitoneal administration of IMMUFLAM from day 14 to day 35 causes an increase in the threshold of mechanical sensitivity to a value of 18.28±0.65 s (day 30), compared with the animals treated with only the vehicle (11.85±0.61 s) (P≦ 0.001). This effect lasts until day 35 corresponding to the end of the treatment. Hargreaves test on rats treated with paclitaxel (groups A and B) also showed a notable reduction in reaction time to thermal stimulus (respectively 9.18±0.65 s and 9.73±0.43 s on day 10), indicating the beginning of a hyperalgesic state. This state of thermal hyperalgesia is still evident in the animals treated with only the vehicle up to day 35 (5.82±0.18 s). In contrast, intraperitoneal administration of IMMUFLAM for twenty-one days significantly increases the threshold of thermal sensitivity with a value equal to 10.21±0.34 (P≦ 0.001).

### 12. Visceral Pain: Acetic Acid Model

We verified the effect of IMMUFLAM on a model of visceral pain. 30 mice were divided into two groups of 15: one group received IMMUFLAM 4 mg/kg intraperitoneally while the other group was treated with only the pharmaceutical vehicle. After the pre-treatment injection, 0.6% acetic acid was administered as a bolus injection 0.3 ml IP. The mice were put in individual observation chambers for assessing, after a period of 20 minutes, the number of contractions induced in each animal following injection of acetic acid. The count started 5 minutes after administration of acetic acid.

The results are presented in the bar chart in FIG. 20.

IMMUFLAM proved to be effective in the treatment of visceral pain.

### 13. Inhibition of Activation of T Cells In Vitro

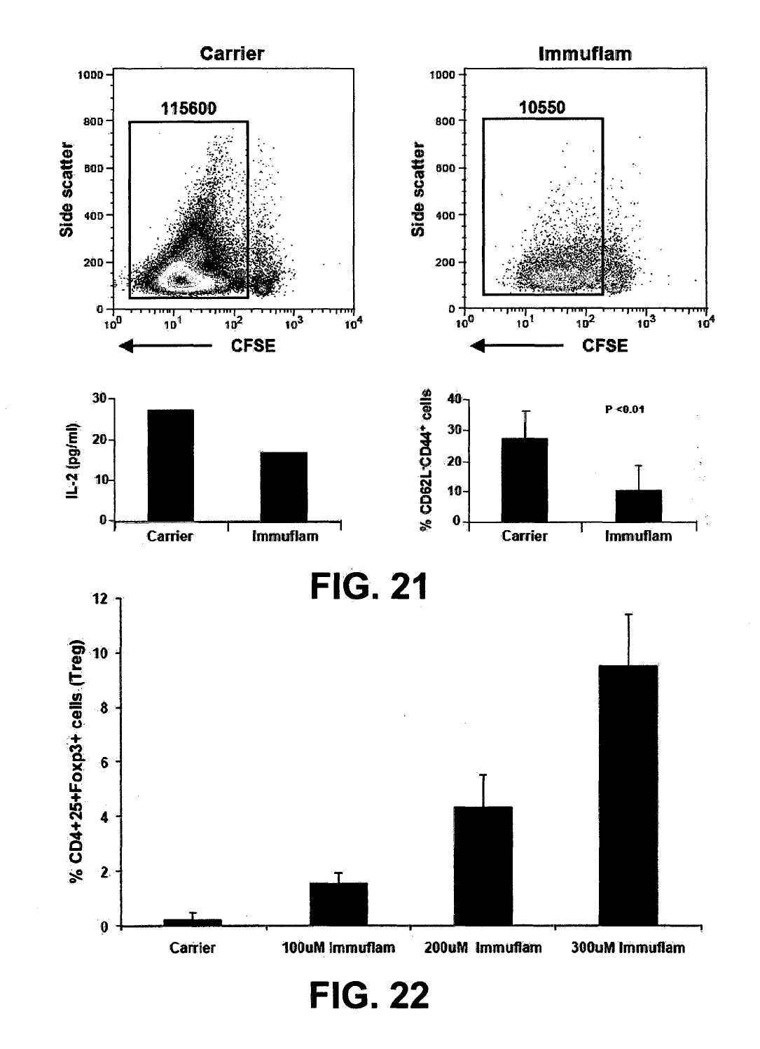

The lymphocytes are cells of the immune defences that direct and amplify the response to attack. Their imbalance or malfunction is also at the root of autoimmune diseases. Successful increase in the ratio in favour of Treg cells can restore the equilibrium of the immune response and combat the resultant disorders. We analysed the activation of isolated naive murine CD4+ cells in the cytofluorimeter for the phenotype CD4+CD62L+CD44−CD25−. The cells were labelled with “5,6-carboxyfluorescein diacetate succinimyl ester” (CFSE) and we evaluated cellular proliferation in quantitative acquisitions with the cytofluorimeter based on the dilution of fluorescence, which depends on the proliferative activity of the cell. FIG. 21 shows the more than ten-fold reduction in the number of proliferating cells (identified by the quadrant inside the dot plot) in the presence of IMMUFLAM at a concentration of 300 μM. The “side scatter” of the cells (a parameter of activation of the CD4 cells) is also reduced by treatment with IMMUFLAM. Moreover, treatment with IMMUFLAM significantly reduces the secretion of interleukin-2 (panel bottom left, values obtained in ELISA in the culture supernatant at 24 h), expression of the marker of activation of CD44 T cells and the “shedding” of L-selectin, CD62L (panel bottom right with percentage of cells positive for CD44 and negative for CD62L). These data demonstrate that IMMUFLAM inhibits the activation of T cells.

### 14. Induction of T Regulatory (T_{reg}) Cells by Naive CD4 Cells

We evaluated the capacity of IMMUFLAM to induce in vitro the differentiation of naive CD4 cells into immunosuppressive T_{reg} cells characterized by the phenotype CD4+CD25+ and by expression of the Foxp3 transcription factor. The bar chart in FIG. 22 shows the induction of T_{reg} cells in vitro starting from naive CD4 cells purified in the cytofluorimeter from a suspension of murine lymph nodes for the phenotype CD4+CD25−CD44−CD62L+.

The naive CD4 cells were stimulated with anti-CD3 antibodies for 96 h in the presence of increasing concentrations of IMMUFLAM. IMMUFLAM induced the differentiation of T_{reg} cells in a manner that was dependent on the dose used.

### 15. Inhibition of Maturation of Dendritic Cells

The dendritic cells (DCs) supply information concerning pathogenic antigens to the other cells of the immune system. They are specialized in presentation of the antigen and are capable of activating the cytotoxic T lymphocytes.

The immature DCs are localized in the non-lymphoid tissues, where they capture the antigen; then they migrate to the secondary lymphoid organs, where they stimulate virgin T lymphocytes, in the meantime becoming mature cells.

The DCs are capable of functioning at very low concentrations, increasing the immune response in various disorders and inducing immune tolerance.

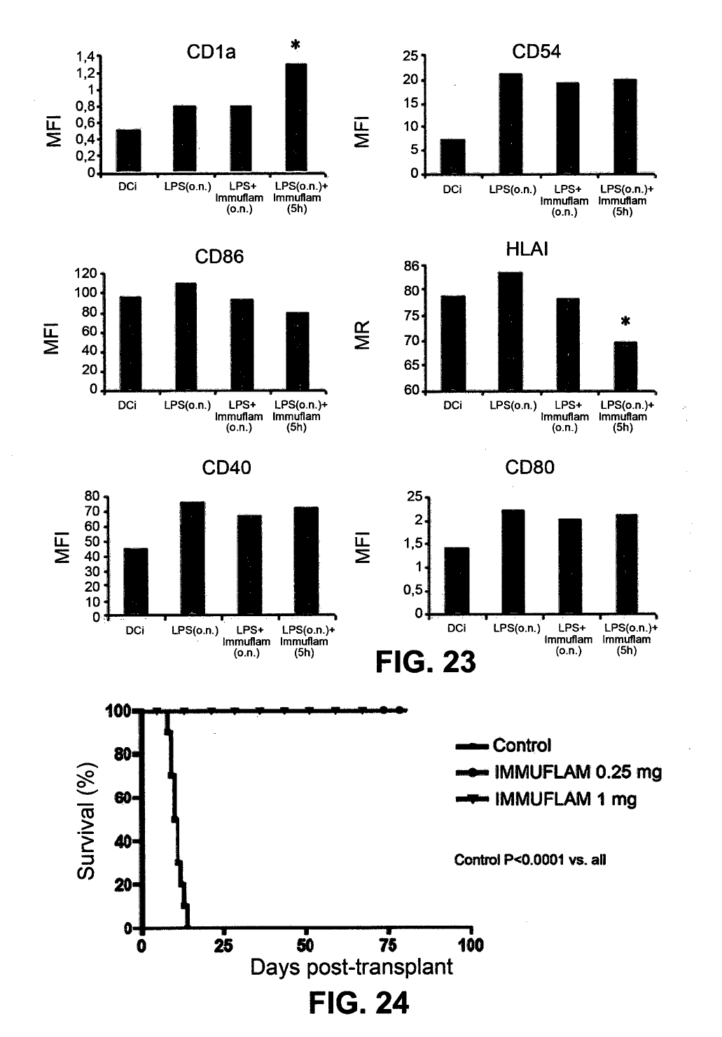

The effect of IMMUFLAM on the various stages of maturation of the DCs was evaluated. The study was carried out using flow cytometry by analysing the principal membrane antigens characterizing the stage of differentiation and maturation of the DCs. The results obtained are shown in FIG. 23.

The DCs were obtained from peripheral whole blood and were plated in a suitable medium (DC medium). The immature DCs (DCi) on day 6 of culture were collected, analysed in the cytofluorimeter and were brought to maturation by stimulation with LPS 10 μg/ml overnight (O.N.). IMMUFLAM 300 μM was added to DC medium during the last 5 hours or simultaneously with maturation of the DCi with LPS (10 μg/ml) overnight. CD1a is a marker of immaturity specific to the DCs, expression of which decreases in mature DCs. It was shown to increase on the DCs matured with LPS and stimulated with IMMUFLAM at 5 hrs relative to the DCs matured with LPS alone.

CD80 and CD86 are fundamental co-stimulatory molecules in the process of antigen presentation, and expression of them generally increases in mature DCs compared to immature DCs.

In this case the action of IMMUFLAM was the only negative regulation of CD86 in DCs matured with LPS. There were no effects on CD80. The class I histocompatibility antigen also decreases following stimulation with IMMUFLAM both overnight and at 5 hours.

The effect of negative regulation of IMMUFLAM was less marked in the modulation of CD54 (adhesion molecule) and CD40 (receptor of T lymphocytes).

It is clear that pharmacological stimulus with IMMUFLAM has an immunosuppressive action, owing to the increase in CD1a+ cells (immature cells) and to the decrease in expression of CD86 and of the type I histocompatibility antigen, the principal “translator” of the type T cytotoxic response.

### 16. Immunosuppressive Effect: Survival of the Allogenic Pancreatic Islets in a Murine Transplant Model

C57BL/6 mice were made diabetic by administration of streptozotocin (170 mg/kg) and, following transplant of allogenic pancreatic islets, were treated with IMMUFLAM.

The pancreatic islets, isolated from the BALB/c mouse, were inoculated in the diabetic mouse according to the procedure described by Abde R et al. (Diabetes 51 (8): 2489-2495). The glycaemia values were measured daily for monitoring the functionality of the transplanted cells.

The significance of rejection of transplanted cells that occurred was attributed to glycaemia values above 250 mg/dl, obtained in two consecutive measurements. After the cells were implanted (day 0), the mice were treated with intraperitoneal injections of 9 mg/kg or 30 mg/kg of IMMUFLAM daily.

In the mice treated with the drug, there is no rejection up to day 100; therefore rejection is considered to be absent (FIG. 24).

17. Immunosuppressive Effect: Survival of the Allogenic Pancreatic Islets, Drug combination

The combination of IMMUFLAM and Rapamycin, currently the most used of the immunosuppressive drugs, was tested on the same animal model.

Treatment of diabetic mice with IMMUFLAM and Rapamycin led to a significant increase in vitality of the pancreatic islets relative to monotherapy with IMMUFLAM or with Rapamycin. Both drugs were administered 6 times (day 0, day 2, day 4, day 6, day 8, day 10); IMMUFLAM was administered at 2 doses.

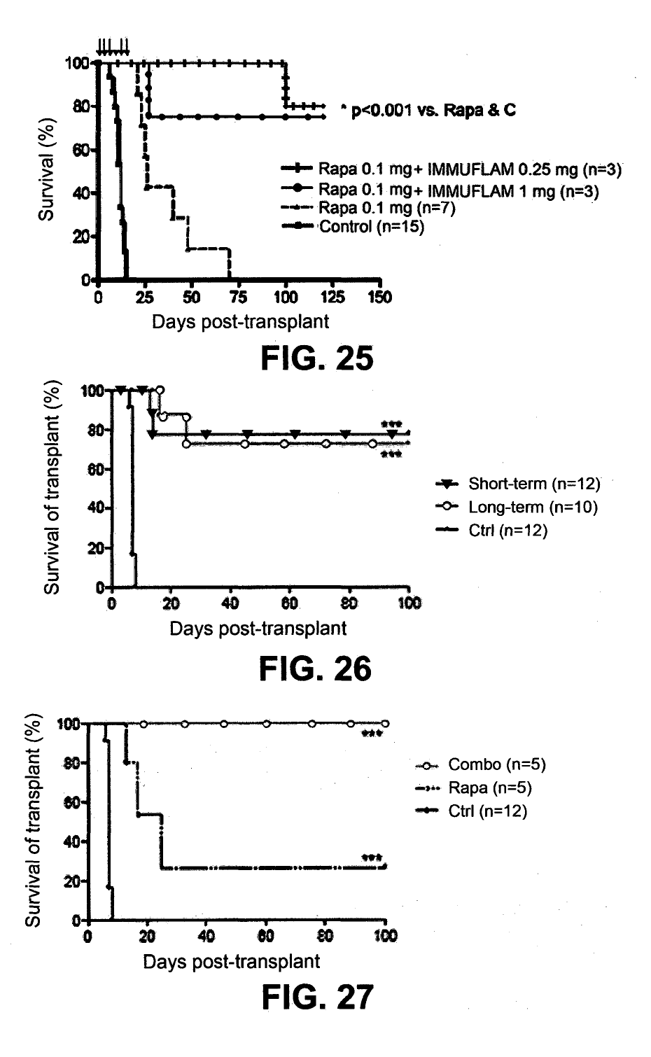

Based on the results obtained (FIG. 25), it could be deduced that IMMUFLAM has a synergistic action, when used with other immunosuppressive drugs, administered in combination, separately or successively with the compound under consideration, and this represents the object of the invention.

### 18. Immunosuppressive Effect: Murine Heart Transplant Model

The immunosuppressive potential of IMMUFLAM was tested on a heterotopic heart transplant model; hearts of BALB/c mice were transplanted into the abdominal cavity of C57BL/6 mice.

The beats of the heart were measured, twice weekly, by palpation; stopping of beating was regarded as a sign of transplant rejection.

The control mice showed rejection in 10 days (Ctrl: mean survival time (MST)=7, n=12). Three different protocols were used for prolonging transplant survival times: the “short-term” protocol (IMMUFLAM 9 mg/kg daily for 14 days), the “long-term” protocol (IMMUFLAM 9 mg/kg every day until rejection), and the “Combo” protocol (IMMUFLAM on alternate days: day 0, 2, 4, 6, 8, 10, 12, 14+Rapamycin 0.1 mg/kg from day 0 to day 10).

In the acute heart transplant models, the hearts of BALB/c were transplanted into the peritoneal cavity of C57BL/6 mice. The control mice invariably reject the transplant in 10 days (Ctrl: MST=7 days, n=12), (FIG. 26). The “short-term” protocol permitted indefinite survival (100 days) in 10 mice out of 12 (n=12; p=0.0001 vs. Ctrl), (FIG. 26). Similarly, the “long-term” protocol permitted indefinite survival in 8 mice out of 10 (n=10; p=0.002 vs Ctrl), and prolonged survival in the remaining 2 (16 days and 25 days; p=0.002 vs. Ctrl), (FIG. 26).

Administration of the drug Rapamycin alone (0.1 mg/kg from day 0 to day 10) ensured indefinite survival in 2 mice out of 5 (MST=25 days, n=5; p=0.0002 vs. Ctrl), (FIG. 27) while the “Combo” significantly permitted indefinite survival of the whole population of mice (n=5; p=0.0001 vs. Ctrl), (FIG. 27). Immuflam administered daily or in combination proves extremely effective in preventing rejection after organ transplants.

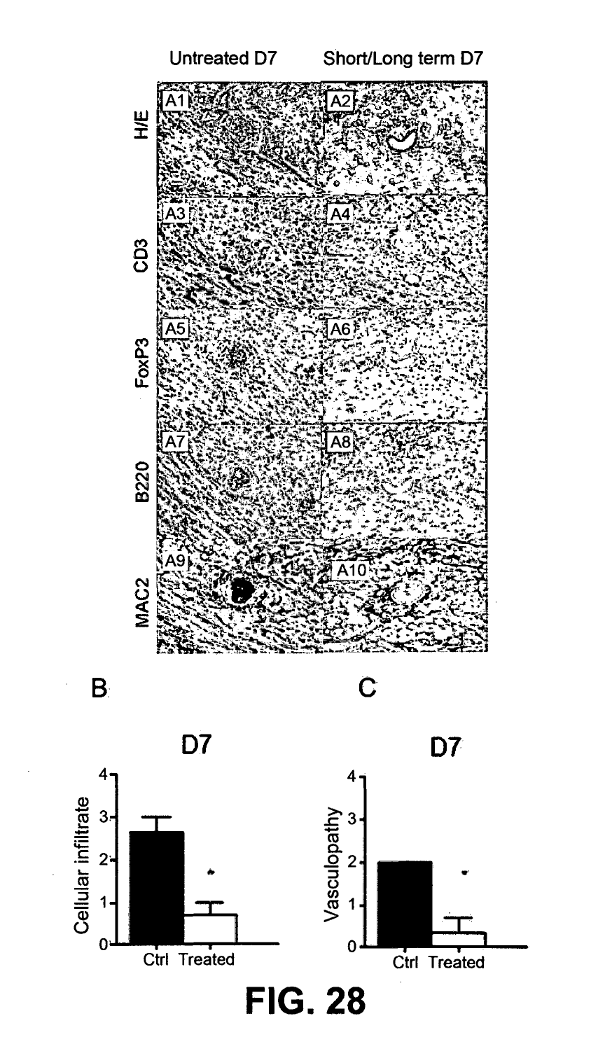

The histological sections of the hearts on day 7 of BALB/c mice, transplanted into a C57BL/6 host, and treated with the “short-term” protocol, were also analysed. Staining with haematoxylin-eosin revealed a significant difference between the control mice and the mice treated with the drug, showing in the first case considerable lymphocyte tissue infiltration, which is absent in the second case (A1-A2 in FIG. 28). The tissues derived from the same model were then labelled with anti-B220 and anti-CD3 monoclonal antibodies, showing similar behaviour: the B and T lymphocytes appear to decrease numerically in the treated mice relative to the controls (A3, A4, A7, A8 in FIG. 28), confirming the efficacy of the dosage of IMMUFLAM provided in the “short-term” protocol. Labelling with anti-FoxP3 monoclonal antibodies did not show significant differences (A5 and A6). However, the presence of mature macrophages is completely inhibited in the mice treated with this protocol (A9 and A10).

Assessing the scores assigned to the different degrees of infiltration, it was significant that the mice treated with IMMUFLAM show presence of cells of the immune system, numerically less than the untreated controls (Ctrl=2.7±0.3 vs. Treated=0.7±0.3, p=0.01) (FIG. 28B). Finally vasculopathy, the most evident symptom of prolonged inflammatory events, was attenuated in the mice treated with IMMUFLAM according to the “short-term” protocol relative to the controls (Ctrl=2.0±0.003 vs. Treated=0.3±0.3, p=0.007) (FIG. 28C).

It should be noted that application of the “combined” protocol of the two drugs permitted further prolongation of survival that led to indefinite survival of the whole population of mice.

These experiments have confirmed the hypothesis that IMMUFLAM does indeed possess anti-inflammatory properties, reducing the production of inflammatory cytokines.

### 19. Immunosuppressive Effect in Rheumatoid Arthritis

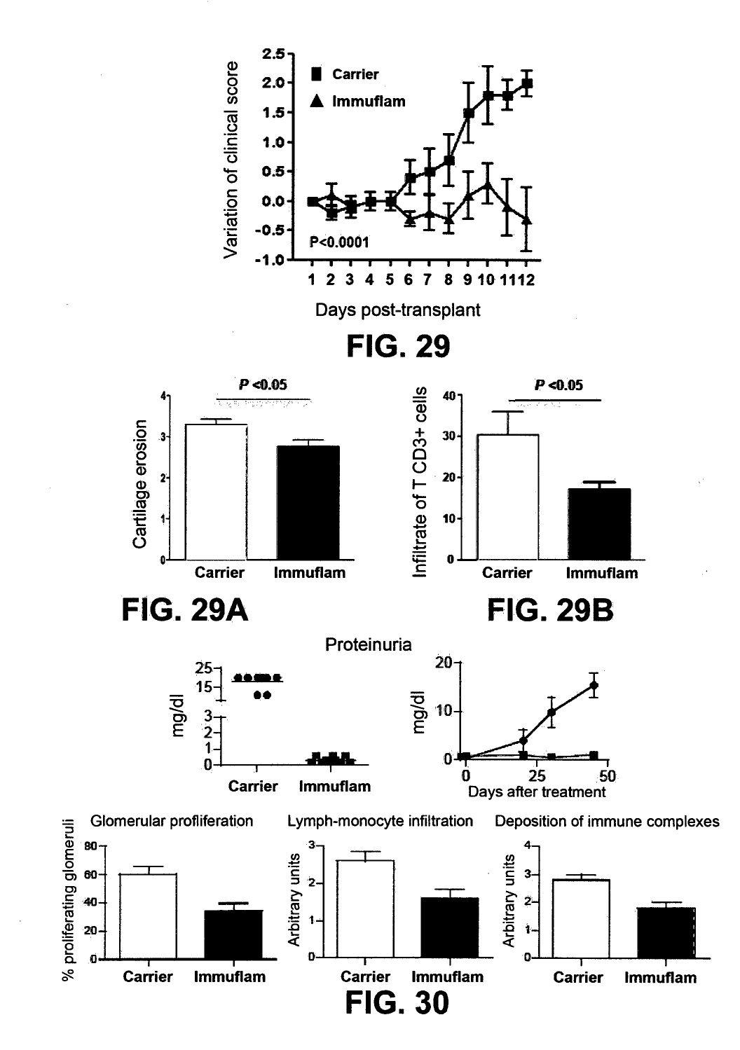

Rheumatoid arthritis (RA) is a chronic inflammatory disease that affects the joints in particular and is manifested by pain, stiffness and synovitis, which lead to destruction of the joint. Immunization of DBA/1J mice with type II collagen (CII) by intradermal injection, at the base of the tail, of 0.2 ml of an emulsion containing 2 mg/ml of bovine CII in complete Freund adjuvant induces T cells and autoantibodies specific to CII with clinical and histopathological signs that reproduce RA. Eighteen to twenty days after immunization, signs of inflammation appear, affecting one or more joints. At the first clinical signs of inflammation (inflammation of the digits and joints) with a score above 1.5 (Camps et al., 2005), we treated the animals with IMMUFLAM intraperitoneally (0.25 mg). Treatment with IMMUFLAM produced a significant reduction in the clinical score relative to the animals treated with the carrier (FIG. 29).

Treatment with IMMUFLAM produced a significant reduction in cartilage erosion measured as content of proteoglycans (FIG. 29A). To establish the degree of infiltration of T cells in the arthritic lesions, sections fixed in formalin and embedded in paraffin were incubated with rabbit polyclonal anti-CD3 antibodies. After detection with peroxidase-labelled secondary antibodies, each sample was evaluated at 40× magnification and 3 immunoreactive “hot spots” were selected for counting the CD3⁺ lymphocytes at 200× magnification. The treatment with IMMUFLAM caused a significant reduction in CD3⁺ lymphocytes infiltrating the joint (FIG. 29B).

### 20. Immunosuppressive Effect: Systemic Lupus Erythematosus (SLE)

Systemic lupus erythematosus (SLE) is a chronic autoimmune disease that generally begins with arthritis or arthralgia. Haematuria or proteinuria may be the only renal symptom, however, lupus glomerulonephritis may lead to acute or terminal renal failure. An experimental model of SLE consists of female F1 mice derived from crossing of the NZB strain with NZW. To evaluate the possible effect of IMMUFLAM in SLE, we treated female NZB/NZW F1 mice with 0.25 mg of IMMUFLAM or carrier 5 days a week for six weeks, starting the treatment when the mice had developed anti-DNA antibodies. During the six weeks of treatment, all the mice treated with carrier developed proteinuria, while in the mice treated with IMMUFLAM, proteinuria did not appear in the two months following the treatment (FIG. 30: proteinuria 60 days after the end of the treatment and panel on right: kinetics). Moreover, the mice treated with IMMUFLAM showed reduced renal injury, with a significant decrease in glomerular proliferation, lymphocyte infiltration and deposition of immune complexes (histopathological “scores” at 60 days from the end of treatment in the bottom panels in FIG. 30). These results demonstrate that IMMUFLAM is able to improve the course of SLE.

### 21. Immunosuppressive Effect in Chronic Intestinal Inflammation (Crohn's Disease and Ulcerative Proctocolitis)

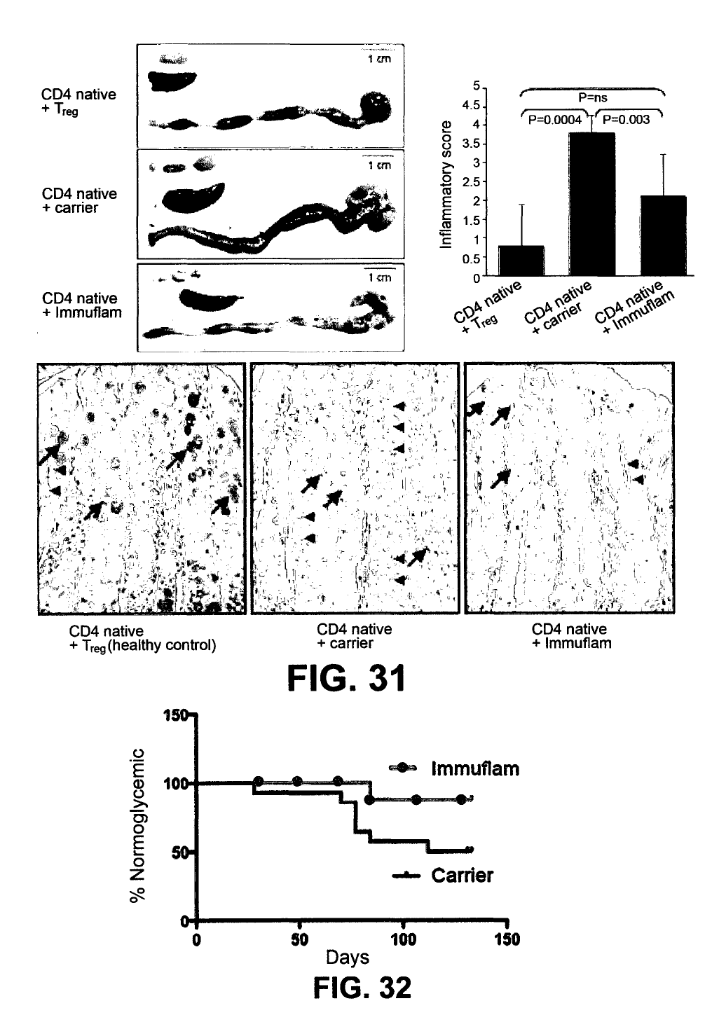

To study the effect of IMMUFLAM in idiopathic inflammatory diseases of the intestine, such as Crohn's disease and ulcerative colitis, we used lymphopenic mice with deletion of cd3ε. T lymphocytes are not present in these animals, and transfer of naive CD4 cells causes chronic intestinal inflammation. The simultaneous transfer of regulatory T cells (T_{reg}) CD4+CD25+ precedes the onset of the disease. FIG. 31 shows the macroscopic appearance of the colon, spleen and mesenteric lymph nodes, representative of each experimental group. Compared to mice reconstituted with naive CD4 and T_{reg} cells (healthy controls), mice reconstituted with naive CD4 cells and treated with carriers show a severe oedematous-haemorrhagic picture of the colon with splenomegaly and lymphadenomegaly. Treatment with IMMUFLAM two weeks after injection of naive CD4 cells gives a dramatic improvement of the inflammation of the colon with presence of formed faeces, spleen and lymph nodes all normal. In mice reconstituted with naive CD4 and T_{reg} cells, staining of colon sections with Alcian-PAS did not reveal inflammatory changes and a large number of muciparous goblet cells can be seen with voluminous droplets positive to Alcian-PAS aligned along the cryptae (arrows). The accumulation of Alcian-positive mucus filaments is visible inside the lumen of the cryptae (arrowheads). In the mice injected with CD4 cells and treated with carriers, muciparous goblet cells have completely disappeared along the cryptae. The cryptae are markedly hyperplasic with intensive cellular crowding, stratification, obliteration of the lumen (arrowheads), and a high frequency of mitotic cells is encountered (arrows). In the mice injected with CD4 cells and treated with IMMUFLAM, in contrast, muciparous goblet cells positive to Alcian-PAS are identifiable along the cryptae and on the mucosal surface (arrows). The cryptae are moderately hyperplasic and mucus filaments can be seen inside the lumen of the cryptae (arrowheads).

Statistical analysis of the histological “scores” based on the classification published by Asseman and co-workers (Asseman et al., 2003) (0: absence of lesions; 1: mild lesions; 2: moderate lesions; 3: severe lesions; 4: extremely severe lesions) identified a significant improvement of the lesions in the animals treated with IMMUFLAM relative to the animals treated with carriers and absence of a significant difference of the lesions in the mouse treated with IMMUFLAM compared to the healthy control (naive CD4+T_{reg} cells) (FIG. 31). These results demonstrate a potent anti-inflammatory activity of IMMUFLAM in idiopathic inflammatory diseases of the intestine, such as Crohn's disease and ulcerative colitis.

### 22. Immunosuppressive Effect in Type 1 Diabetes

NOD (“non-obese diabetic”) mice represent the spontaneous murine model of type 1 diabetes (Adorini et al., 2002). The loss of metabolic control characterized by hyperglycaemia and glycosuria, which defines the disease clinically, is preceded by a long phase called pre-diabetes, during which the response to specific autoantigens of the beta cells of the pancreas develops subclinically, but progressively. In the NOD mice, the disease begins clinically around the 14th week of age when the infiltrating inflammatory cells invade the islets and develop an aggressive insulitis, which leads to destruction of the beta cells and causes hyperglycaemia (Anderson and Bluestone, 2005). The autoreactive CD4+ and CD8+T cells play a fundamental role in the pathogenesis of type 1 diabetes.

We treated female NOD mice at 11 weeks of age with 0.25 mg IMMUFLAM or carriers intravenously for 5 days every two weeks and monitored glycaemia every week. Only one out of 8 mice treated with IMMUFLAM developed diabetes at the 28th week of age, whereas 7 out of 14 mice treated with carriers developed diabetes or died starting from the 14th week of age (FIG. 32). This result demonstrates that administration of IMMUFLAM is able to prevent full-blown diabetes in NOD mice.

### 23. Efficacy of IMMUFLAM in the Treatment of Muscular Dystrophy

Muscular dystrophia is a term that comprises a group of serious neuromuscular diseases of a degenerative nature, which are genetically determined and cause progressive atrophy of the skeletal musculature, such as Becker muscular dystrophy, Duchenne muscular dystrophy, limb-girdle muscular dystrophy, mutations of the gene of dysferlin (a sarcolemma protein) that cause limb-girdle muscular dystrophy of type 2B and Miyoshi myopathy. They are hereditary disorders of infancy caused by mutations of the dystrophin gene. This protein forms part of a multiprotein complex that connects the cytoskeleton of a muscle fibre to the extracellular matrix via the plasma membrane. In patients with MD, loss of dystrophin makes the muscle cell plasma membrane more fragile and susceptible to damage induced by contraction. Inflammation in muscle tissue with dystrophin deficit plays an important role in promoting muscular injury. For this reason, corticosteroids are still the only therapeutic agent currently used in patients with MD. The commonest form is Duchenne muscular dystrophy (DMD).

The mdx mouse is characterized by a nonsense mutation in the dystrophin gene and constitutes an experimental model of DMD. The muscle of mdx is histologically normal in early postnatal development, but goes towards necrosis starting from the third week of life.

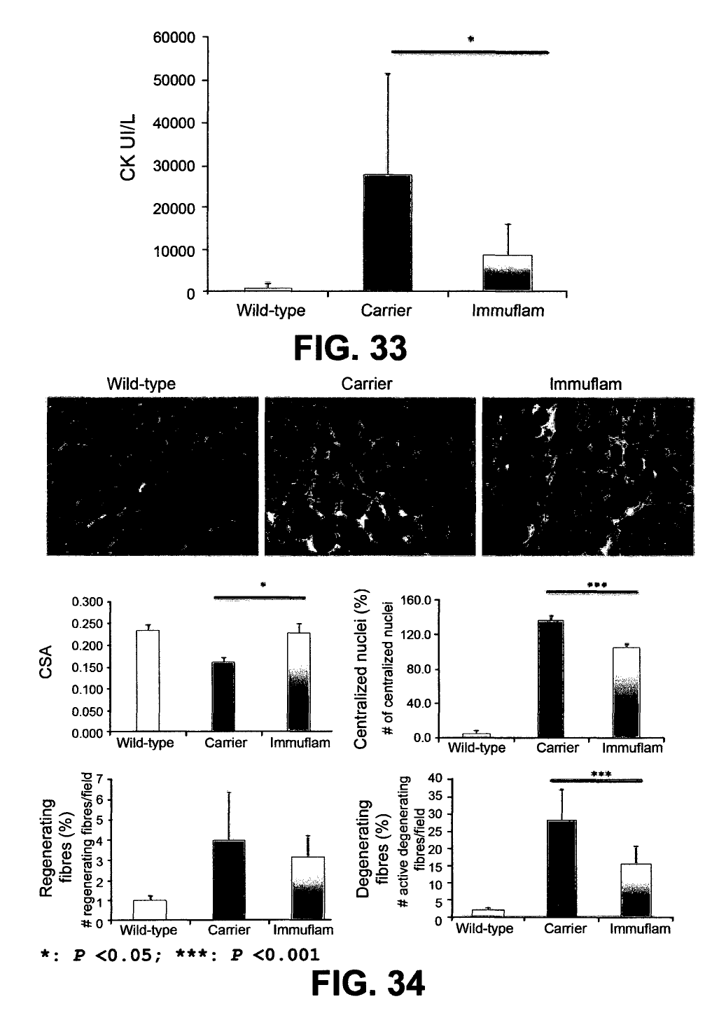

The mdx mice show high levels of creatine kinase (CK) in the serum and accumulation of inflammatory cells, both markers of muscular degeneration. To assess the effect of IMMUFLAM in the progression of dystrophy and of inflammation in DMD, we treated mdx mice (n=10) at 4 weeks of age (in accordance with the recent SOPs for preclinical drug trials in mdx) with IMMUFLAM (0.25 mg/mouse) intraperitoneally every day for two weeks. The same number of mice of the same age was treated with the vehicle as control. After two weeks of treatment with IMMUFLAM, the CK levels had decreased by 70% (P<0.05) (FIG. 33).

Cross sections of anterior tibial muscles stained with haematoxylin/eosin were quantified in duplicate in blind conditions with ImageJ software. In the same sections we also assessed centralization of the nucleus, degeneration of muscle fibres and their regeneration. The untreated mdx mouse showed all the pathognomonic characteristics of DMD such as increased variability in size of the fibres, presence of degenerative/necrotic fibres, increased regeneration, nuclear centralization and inflammatory infiltrates. Treatment with IMMUFLAM led to a significant increase in area of the muscle cross sections (CSA, “cross sectional area” in FIG. 34) relative to the animals treated with the carrier. The values obtained for the mice treated with IMMUFLAM were similar to those obtained for the control mice of the same age and genotype but without mutation in the dystrophin gene. Moreover, we also found significant reductions both in the number of degenerative fibres and in centralization of the nucleus.

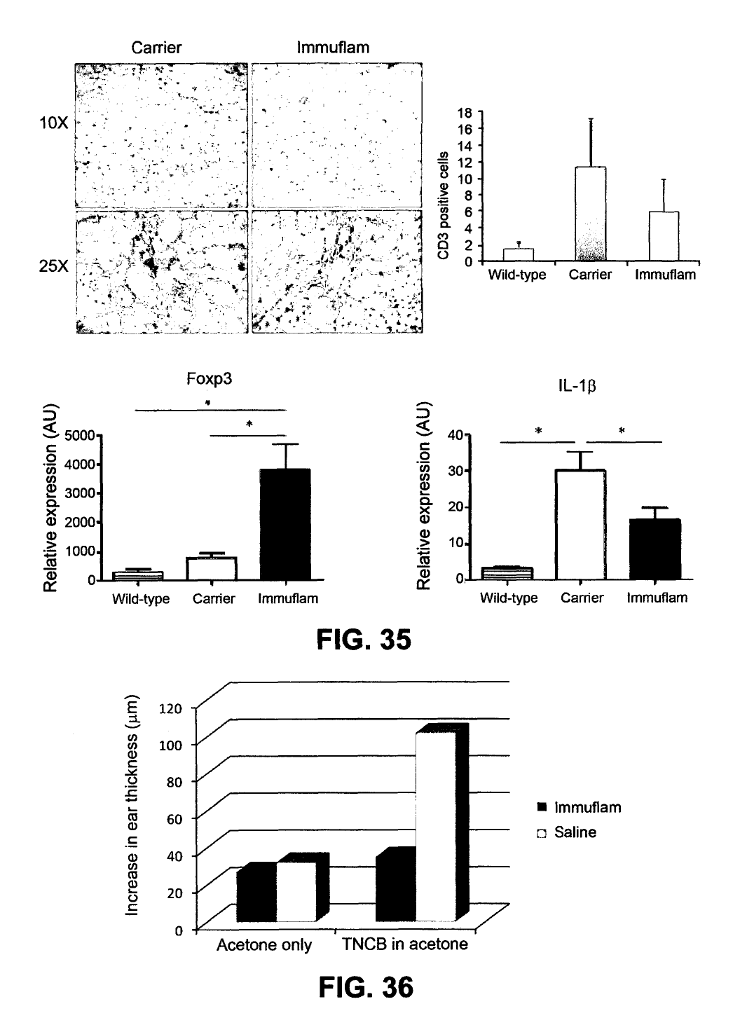

To assess the effect of IMMUFLAM on the inflammatory infiltrate in the muscle of mdx mice, the muscle sections were decorated with anti-CD3 antibodies (T cells). This analysis detected a 40%) decrease in the number of cells positive for CD3. In addition to the significant reduction in the infiltration of CD3⁺ cells in the muscle, IMMUFLAM caused a significant increase in the levels of mRNA typical of the T_{reg} cells and a significant reduction in II-1β, measured by quantitative PCR, compared to mice treated with the vehicle (bottom panels in FIG. 35). These data demonstrate that IMMUFLAM reduces muscular degeneration and inflammation in the mdx mouse; moreover, it has proved capable of inducing immunosuppressive T_{reg} cells in skeletal muscle.

### 24. Allergic Contact Dermatitis

Allergic contact dermatitis (ACD or allergic eczema) is an inflammatory (immune) reaction of the skin, caused by hyperreactivity of the immune system to a particular substance present in the environment. The commonest symptoms of CD are those typical of all dermatitides: oedema, reddening and pruritus. It is a skin disease that is not contagious, but is troublesome and painful.

We wished to test the activity of IMMUFLAM in an animal model of this disorder.

C57BL/6 WT mice are sensitized by applying, on day 0, 100 μl of 3% TNCB (2,4,6-trinitrochlorobenzene) in acetone, or only acetone as control, on the skin of the abdomen.

As stress, 20 μl of 1% TNCB is applied on the skin of the back of both ears on day 5. This stress causes an allergic reaction that is manifested as swelling and reddening. The thickness of the ear (symptom of swelling) is measured 24 hours after application of the stimulus, using callipers (Mitutoyo). IMMUFLAM was injected locally in the auricle at a dose of 2 mg/kg 4 hours before the sensitizing stimulus.

As can be seen from the bar chart in FIG. 36, application of IMMUFLAM was able to inhibit the allergic reaction due to the sensitizing stimulus.

### 25. Graft-Versus-Host Disease

Graft-versus-host disease (GvHD) is a complication, often with fatal outcome, of allogenic transplantation of haematopoietic cells (allo-HCT). GvHD is due to the donor T lymphocytes, which are activated against the host into which they have been transplanted, because they recognize the allo-antigens of the host as foreign.

Two groups of NOD/scid mice of 8-10 weeks were used, and were kept in cages under sterile flow at an SPF animal facility.

On day −1, to avoid residual activity of natural killer cells, the mice were administered an anti-NK antibody intraperitoneally (TMβ-1.1 mg/mouse). On day 0 the mice were irradiated (300 rad) and they were administered, intraperitoneally, 20×10⁶ human peripheral blood mononuclear cells (PBMCs), previously isolated. The PBMCs are isolated by means of buffy coat from blood from healthy donors.

On the same day, the mice were treated with IMMUFLAM intraperitoneally at two doses, mg/kg and 20 mg/kg. The treatment was repeated daily for 15 days.

The mice were checked regularly 3 times a week to monitor the weight loss (also as independent variable) and signs of GvHD using a clinical score (0 for weight loss < 10%, 1 for 10%-20%, 2 for 20%; hunching (0-2), reactivity (0-2), appearance of the fur (0-2), and skin integrity (0-2)), with a maximum score of 10.

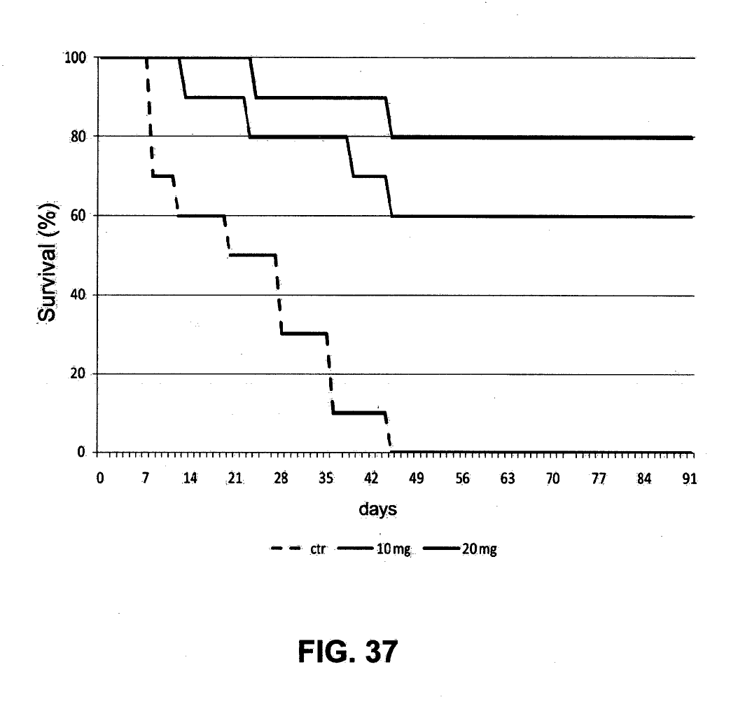

As can be seen in the diagram in FIG. 37, the untreated mice develop GvHD early and they all die within 50 days (natural death or compassionate euthanasia in the case of weight loss >20%). The mice treated with IMMUFLAM develop statistically significant, dose-dependent resistance to GvHD, reaching three months from transplant, beyond which point they are considered to be out of danger.

### 26. Asthma

Asthma is a chronic inflammatory disorder, characterized by variable airway obstruction, excessive production of mucus and hypersensitivity to non-specific stimuli. The inflammatory process is orchestrated by eosinophils, mast cells, Th2 lymphocytes and in particular by the dendritic cells.

Sensitization of mice with ovalbumin (OA) adsorbed on aluminium hydroxide, administered intraperitoneally on day 0 and day 7, can cause a similar asthmatic manifestation when the mouse is subsequently (days 19-21) challenged with OA aerosol. The mice were anaesthetized 30 minutes before the OA aerosol and the treated group received an intratracheal injection of IMMUFLAM 6 mg/kg.

Twenty-four hours after the last administration of OA, a bronchoalveolar lavage was carried out with 3×1 ml of PBS free from calcium and magnesium ions.

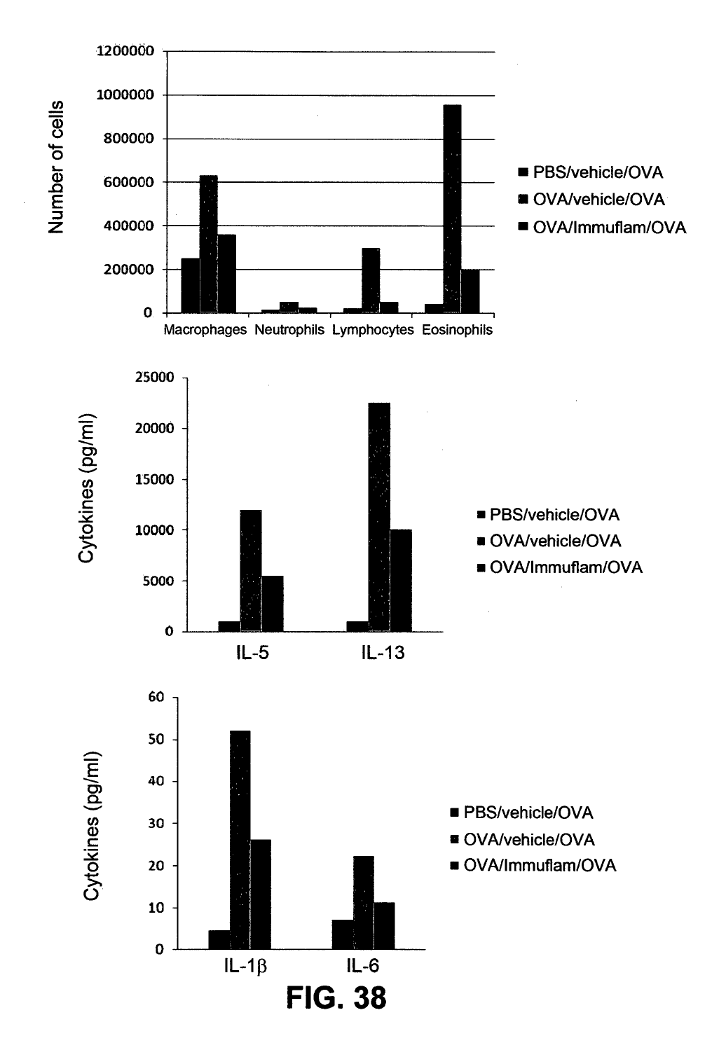

Massive presence of eosinophils, neutrophils, lymphocytes and macrophages can be detected in the bronchoalveolar fluid (BALF) when an allergic reaction has been triggered. Significantly lower levels of immune cells and mediators of inflammation were found in the BALF of the mice treated beforehand with IMMUFLAM, as shown in the bar charts in FIG. 38.

The group not sensitized with OA, but treated with aerosol, display the reference physiological values of the cells and of the inflammatory cytokines.

### 27. COPD

Chronic obstructive pulmonary disease (COPD) is a degenerative disorder of the respiratory system. As a result of repeated stimulus, an inflammatory process develops, accompanied by production of mucus and by obstruction of the respiratory tree, which induce changes in the pulmonary parenchyma and eventually development of emphysema and/or chronic bronchitides.

The aetiology is still unknown, but genetic predisposition and an environmental stimulus such as smoking, active or passive, or exposure to environmental pollution, undoubtedly play a large part.

C57/B1/6 mice aged 6-8 weeks were exposed to room air or to the smoke of 5 cigarettes/day (Virginia brand with filter; tar 12 mg and nicotine 0.9 mg) for 3 consecutive days. For exposure to smoke, a special cage was used, which permits air exchange with the exterior or with the device that contains the cigarette only due to a mechanical fan, which also allows the smoke to be diluted with the external air (1:8) in order to simulate the situation of a smoker.

Starting on the fourth day, the mice were anaesthetized 30 minutes before exposure to the smoke and were administered 6 mg/kg of IMMUFLAM or vehicle by intratracheal injection. The mice were sacrificed one hour after the end of the treatment and a bronchoalveolar lavage was carried out.

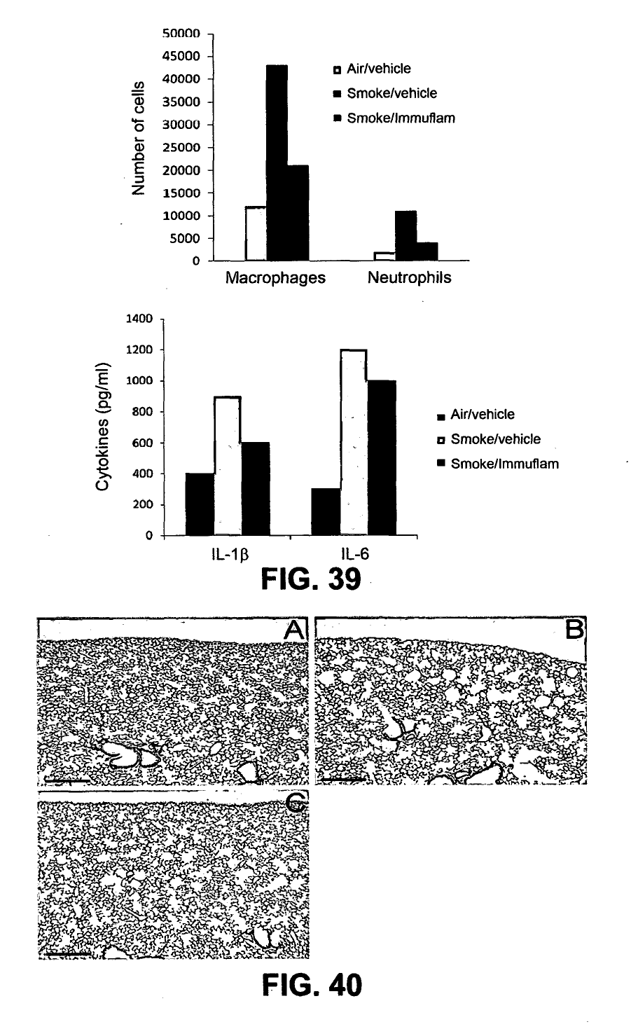

The administration of IMMUFLAM caused a slight finding of immune cells and of messengers of inflammation in the BALF, indicating a definite slowing of the inflammatory effect (FIG. 39).

The activity of IMMUFLAM was also tested in a model of chronic exposure, which is closer to the aetiology of the human disorder. The mice were exposed to the smoke of 3 cigarettes a day or air for 5 days a week for 6 months, and were treated 30 minutes before the daily exposure with IMMUFLAM 4 mg/kg i.p.

At the end of the 6 months, the mice were sacrificed and a morphometric and histologic examination was conducted on the lung tissue.

The mean interalveolar distance (Lm) and mean internal surface area of the lung (USA) were measured, being parameters that are correlated with degeneration of lung tissue and with the resultant decrease in respiratory function.

| | Lm (μm) | ISA (cm²) | Histologic group |
|---|---|---|---|
| Air/vehicle | 35.3 ± 0.7 | 1195 ± 41 | A |
| smoke/vehicle | 40.3 ± 0.9 | 1089 ± 36 | B |
| smoke/IMMUFLAM | 36.3 ± 1.1 | 1153 ± 59 | C |

Histologic examination shows that the lung tissue of the treated mice is similar to that of the mice exposed to purified air (FIG. 40), the alveolar dimensions are similar (A and B) and the number of alveoli is higher.

The untreated group, exposed to smoke, shows foci of emphysema where parts of the alveoli have been destroyed (C, empty white areas).

IMMUFLAM proved to be active both in counteracting the inflammatory process underlying COPD, and in slowing, if not arresting, the degenerative process of the lung tissue underlying the loss of respiratory function that is typical of the disorder.

### 28. Cystic Fibrosis

Cystic fibrosis (CF) is a hereditary genetic disease that mainly affects the respiratory and digestive systems of children and young adults.

In cystic fibrosis, the abnormal contact surface of the airways causes retention of inhaled bacteria and consequent inflammation. Dense, viscous mucus is produced, which obstructs the airways, causing persistent endobronchial inflammation and permanent lung damage due to residual fibrotic scarring.

Airway inflammation is manifested early in the patient's life and is out of proportion to the inflammatory stimulus (e.g. microbial infection).

For the experiments, 32 mice were used with the R117H CFTR(CF) mutation, responsible for the onset of the inflammatory disorder, and 18 wild-type (WT) mice with a genetic background of the C57BL/6 type.

The trachea was then intubated with a MicroSprayer (PennCentury), capable of vaporizing the drug as aerosol at a concentration of 25 μl of IMMUFLAM 300 μM or of vehicle. The mice were weighed daily.

On day 4, all the mice received the inflammatory stimulus (1 μg of LPS) together with IMMUFLAM or vehicle; separate experiments were conducted, inducing only the inflammatory stimulus in the mice. About 24 hours after administration of LPS, the mice were sacrificed and samples of BALF were collected, from which the cellular component was isolated, and was analysed under the microscope for characterizing its leukocyte phenotype.

### Results

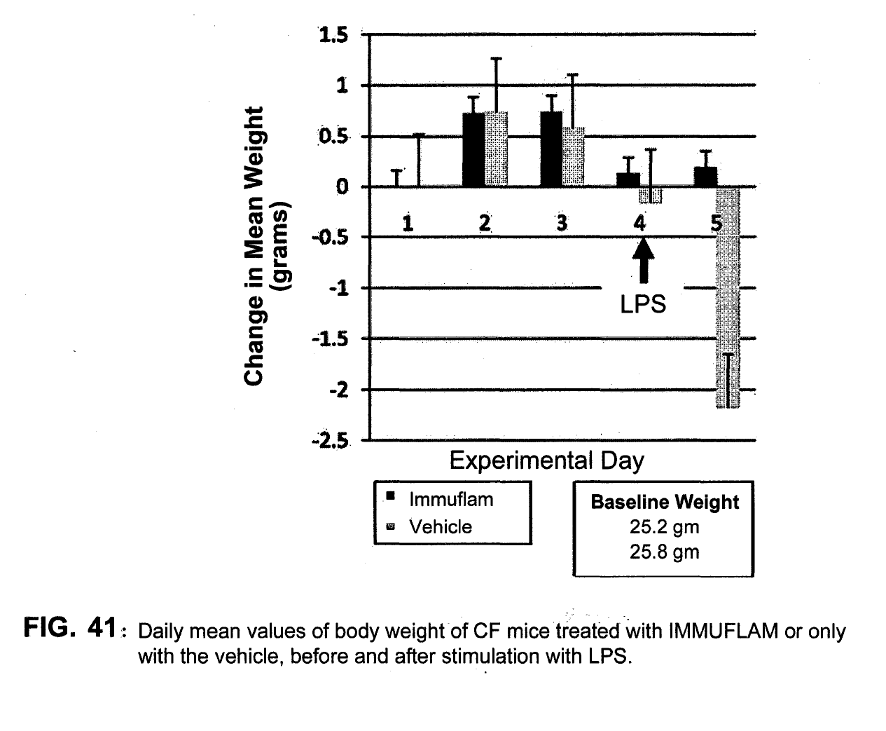

The first index of overall health of the animal was the body weight: the mice treated with IMMUFLAM showed no change in their body weight, whereas the mice treated with the vehicle and the WT mice lost 4-8% and 5% of body weight respectively within the first 24 hours (p< 0.001) (FIG. 41).

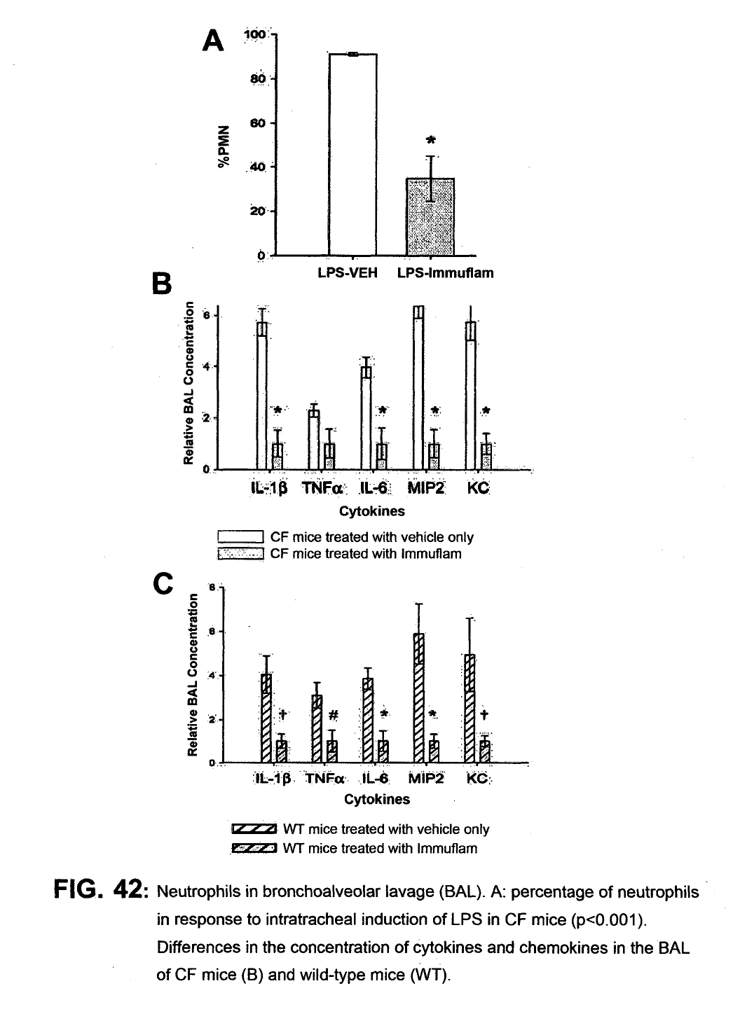

The CF mice treated with IMMUFLAM and stimulated with intratracheal LPS showed a 60% decrease in neutrophil percentage relative to the animals treated with only the vehicle (p<0.001; FIG. 42A). The WT mice treated with IMMUFLAM and stimulated with intratracheal LPS had a 47% decrease in the percentage of neutrophils compared to 89% for the animals treated with only the vehicle (p<0.01).

For further examination of the effect of IMMUFLAM on inflammatory signalling in vivo, the concentrations of cytokines present in the BAL were measured. IL-1β, IL-6, TNFα and the murine analogue of IL-8 showed a reduction in concentrations of 56%-83%, statistically significant, in CF and WT mice that received IMMUFLAM before intratracheal induction with LPS (FIGS. 42B and C).

These results demonstrate a potent anti-inflammatory effect of IMMUFLAM in the preclinical models of cystic fibrosis, suggesting that it can be used therapeutically in lung disorders.

1. The compound 2-[1-(6-aminopurin-9-yl)-2-oxo-ethoxy]prop-2-enal of formula: 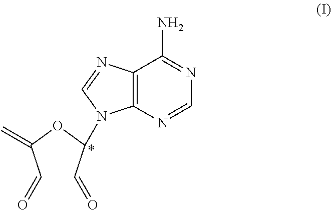 [SMILES: C=C(C=O)OC(C=O)n1cnc2c(N)ncnc21]
its monohydrate of formula (II): 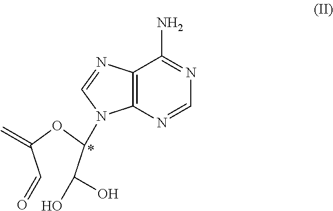 [SMILES: C=C(C=O)OC(C(O)O)n1cnc2c(N)ncnc21]
or its corresponding cyclic monohydrate of formula: 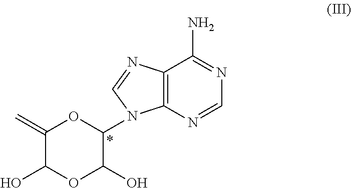 [SMILES: C=C1OC(n2cnc3c(N)ncnc32)C(O)OC1O]
wherein the stereochemical chiral centre indicated with an asterisk can be R (Rectus), S (Sinister) or racemic, including tautomers of the adenosine ring and its pharmaceutically acceptable salts, for use in the treatment of inflammatory pathologies or pain pathology.

2. The compounds according to claim 1, wherein the pain pathology is selected from nociceptive pain (osteoarticular, somatic and visceral), neuropathic pain, cancer pain and pain cine to administration of chemotherapeutic agents.

3. A method of using the compounds according to claim 1, comprising treating pain pathologies, in combined therapy with opioids and/or chemotherapeutic agents.

4. A method of using the compounds according to claim 1, comprising treating immune system pathologies, particularly autoimmune pathologies.

5. A method of using the compounds according to claim 1, comprising therapeutically treating a pathology selected from the group consisting of asthma, COPD, cystic fibrosis, rheumatoid arthritis, diabetes of types I and II, systemic lupus erythematosus (SLE), multiple sclerosis, amyotrophic lateral sclerosis, scleroderma, Sjögren's syndrome, dermatomyositis, polymyositis, Erdheim-Chester syndrome, psoriasis, allergic contact dermatitis, inflammatory bowel disease (IBD), Crohn's disease, Takayasu's disease, transplant rejection, graft-versus-host disease, muscular dystrophy, Duchenne muscular dystrophy or Becker muscular dystrophy, limb-girdle muscular dystrophies (limb-girdle muscular dystrophy of type 2B and Miyoshi myopathy), uveitis, and Alzheimer's disease.

6. A method of using the compounds according to claim 1, comprising treating ocular pathologies selected from the group consisting of diabetic retinopathy, macular degeneration, proliferative vitreoretinopathy, glaucoma.

7. A method of using the compounds according to claim 1, comprising therapeutically treating atherosclerotic processes.

8. A method of using the compounds according to claim 1, comprising administering said compound to a human being, in an amount from 0.01 mg to 20 mg per kg of body weight.

9. A method of using the compounds according to claim 1, comprising treating in vivo or in vitro, of blood or its derivatives in order to induce the generation of regulatory T lymphocytes.

10. A method of using the compounds according to claim 1, comprising treating blood or its derivatives in order to maintain an immature phenotype in dendritic cells.

11. A method of using the compounds according to claim 1, comprising treating with the compound in a pharmaceutical formulation selected from the group consisting of tablet, capsule, gum, candy, powder, solution, emulsion, syrup, cream, ointment, suppository, patch, suitable for administration as a drug.

12. A method of using the compounds according to claim 1, comprising administering by the parenteral (intramuscular, subcutaneous, intradermal, intra-articular, intravenous, intra-arterial), transdermal, oral, sublingual, intranasal aerosol, or rectal route.

13. A method of using the compound according to claim 1, comprising preparing a medicament for therapeutic treatment.
